SEQUENCE LISTING (52 sequences)

SEQ ID NO: 1 - protein, 107 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 2 - protein, 11 aa, Artificial Sequence
  Synthetic
RASQSVSSYLA

SEQ ID NO: 3 - protein, 7 aa, Artificial Sequence
  Synthetic
DASNRAT

SEQ ID NO: 4 - protein, 9 aa, Artificial Sequence
  Synthetic
QQRSNWPLT

SEQ ID NO: 5 - protein, 214 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 120 aa, Artificial Sequence
  Synthetic
QVQLVQSGAEVKKTGSSVKVSCKASGYTFTYRYLHWVRQAPGQALEWMGWITPFNGNTNYAQKFQDRVTITRDRSMSTAYMELSSLRSEDTAMYYCARSGPRGDYVLDYWGQGTLVTVSS

SEQ ID NO: 7 - protein, 7 aa, Artificial Sequence
  Synthetic
GYTFTYR

SEQ ID NO: 8 - protein, 6 aa, Artificial Sequence
  Synthetic
TPFNGN

SEQ ID NO: 9 - protein, 11 aa, Artificial Sequence
  Synthetic
SGPRGDYVLDY

SEQ ID NO: 10 - protein, 450 aa, Artificial Sequence
  Synthetic
QVQLVQSGAEVKKTGSSVKVSCKASGYTFTYRYLHWVRQAPGQALEWMGWITPFNGNTNYAQKFQDRVTITRDRSMSTAYMELSSLRSEDTAMYYCARSGPRGDYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 113 aa, Artificial Sequence
  Synthetic
DVVMTQSPLSLPVTPGEPASISCRSSQSLLQSNGYNYVEWFLQKPGQSPQLLIYLGSYRASGVPDRFSGSGSGTDFTLKISRVEAEDVGVYYCMQGTHWPLFTFGPGTKVDIK

SEQ ID NO: 12 - protein, 16 aa, Artificial Sequence
  Synthetic
RSSQSLLQSNGYNYVE

SEQ ID NO: 13 - protein, 7 aa, Artificial Sequence
  Synthetic
LGSYRAS

SEQ ID NO: 14 - protein, 10 aa, Artificial Sequence
  Synthetic
MQGTHWPLFT

SEQ ID NO: 15 - protein, 220 aa, Artificial Sequence
  Synthetic
DVVMTQSPLSLPVTPGEPASISCRSSQSLLQSNGYNYVEWFLQKPGQSPQLLIYLGSYRASGVPDRFSGSGSGTDFTLKISRVEAEDVGVYYCMQGTHWPLFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 119 aa, Artificial Sequence
  Synthetic
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLDYSLWFDPWGQGTLVTVSS

SEQ ID NO: 17 - protein, 7 aa, Artificial Sequence
  Synthetic
GFTFSSY

SEQ ID NO: 18 - protein, 6 aa, Artificial Sequence
  Synthetic
SYDGSN

SEQ ID NO: 19 - protein, 10 aa, Artificial Sequence
  Synthetic
DLDYSLWFDP

SEQ ID NO: 20 - protein, 449 aa, Artificial Sequence
  Synthetic
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLDYSLWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 110 aa, Artificial Sequence
  Synthetic
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIK

SEQ ID NO: 22 - protein, 14 aa, Artificial Sequence
  Synthetic
RSSTGAVTTSNYAN

SEQ ID NO: 23 - protein, 7 aa, Artificial Sequence
  Synthetic
GANKRAP

SEQ ID NO: 24 - protein, 9 aa, Artificial Sequence
  Synthetic
ALWYSNLWV

SEQ ID NO: 25 - protein, 560 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKGGGGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 26 - protein, 125 aa, Artificial Sequence
  Synthetic
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSS

SEQ ID NO: 27 - protein, 7 aa, Artificial Sequence
  Synthetic
GFTFNTY

SEQ ID NO: 28 - protein, 8 aa, Artificial Sequence
  Synthetic
RSKYNNYA

SEQ ID NO: 29 - protein, 14 aa, Artificial Sequence
  Synthetic
HGNFGSSYVSYFAY

SEQ ID NO: 30 - protein, 584 aa, Artificial Sequence
  Synthetic
QVQLVQSGAEVKKTGSSVKVSCKASGYTFTYRYLHWVRQAPGQALEWMGWITPFNGNTNYAQKFQDRVTITRDRSMSTAYMELSSLRSEDTAMYYCARSGPRGDYVLDYWGQGTLVTVSSGGGSSGGGGSGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 31 - protein, 566 aa, Artificial Sequence
  Synthetic
DVVMTQSPLSLPVTPGEPASISCRSSQSLLQSNGYNYVEWFLQKPGQSPQLLIYLGSYRASGVPDRFSGSGSGTDFTLKISRVEAEDVGVYYCMQGTHWPLFTFGPGTKVDIKGGGGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 32 - protein, 583 aa, Artificial Sequence
  Synthetic
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLDYSLWFDPWGQGTLVTVSSGGGSSGGGGSGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 33 - protein, 15 aa, Artificial Sequence
  Synthetic
GGGGSGGGGSGGGGS

SEQ ID NO: 34 - protein, 15 aa, Artificial Sequence
  Synthetic
GGGSSGGGGSGGGGS

SEQ ID NO: 35 - DNA, 27 bp, Artificial Sequence
  Synthetic
agatgccagatgtgaaattgtgttgac

SEQ ID NO: 36 - DNA, 68 bp, Artificial Sequence
  Synthetic
attttgagctcttaacactctcccctgttgaagctctttgtgacgggcgaggacaggccctgatgggt

SEQ ID NO: 37 - DNA, 33 bp, Artificial Sequence
  Synthetic
actacaggtgtccactcccaggtgcagctggta

SEQ ID NO: 38 - DNA, 26 bp, Artificial Sequence
  Synthetic
acaagatttgggctcaactttcttgt

SEQ ID NO: 39 - DNA, 34 bp, Artificial Sequence
  Synthetic
agttgagcccaaatcttgtgacaaaactcacaca

SEQ ID NO: 40 - DNA, 31 bp, Artificial Sequence
  Synthetic
acgcggatccttatttacccggggacaggga

SEQ ID NO: 41 - DNA, 27 bp, Artificial Sequence
  Synthetic
agatgccagatgtgatgttgtgatgac

SEQ ID NO: 42 - DNA, 35 bp, Artificial Sequence
  Synthetic
actacaggtgtccactccgaggtgcagctggtgga

SEQ ID NO: 43 - DNA, 41 bp, Artificial Sequence
  Synthetic
gtgtaagcttaccatgggtgtgcccactcaggtcctggggt

SEQ ID NO: 44 - DNA, 41 bp, Artificial Sequence
  Synthetic
gtgttctagagccgccaccatggaatggagctgggtctttc

SEQ ID NO: 45 - DNA, 34 bp, Artificial Sequence
  Synthetic
actacaggtgtccactccgatgttgtgatgactc

SEQ ID NO: 46 - DNA, 28 bp, Artificial Sequence
  Synthetic
gggggatcctttgatatccactttggtc

SEQ ID NO: 47 - DNA, 35 bp, Artificial Sequence
  Synthetic
ggcttacagatgccagatgtgaggtgcagctggtg

SEQ ID NO: 48 - DNA, 33 bp, Artificial Sequence
  Synthetic
gatagagctccctccacctgaggagacggtgac

SEQ ID NO: 49 - DNA, 32 bp, Artificial Sequence
  Synthetic
actacaggtgtccactccgaaattgtgttgac

SEQ ID NO: 50 - DNA, 25 bp, Artificial Sequence
  Synthetic
gggggatcctttgatctccaccttg

SEQ ID NO: 51 - DNA, 36 bp, Artificial Sequence
  Synthetic
ggcttacagatgccagatgtcaggtgcagctggtac

SEQ ID NO: 52 - DNA, 36 bp, Artificial Sequence
  Synthetic
gatagagctccctccacctgaagagacggtgaccag

(19) United States
(12) Patent Application Publication
(10) Pub. No.: US 2024/0309116 A1
(43) Pub. Date: Sep. 19, 2024

(54) ANTIBODIES AGAINST ROR1 AND USES THEREOF

(71) Applicant: Zhejiang Shimai Pharmaceutical Co., Ltd., Hangzhou, Zhejiang (CN)

(72) Inventors: Zuoxiang Xiao, Hangzhou, Zhejiang (CN); Jiaping Peng, Hangzhou, Zhejiang (CN); Dongwen Zhou, Hangzhou, Zhejiang (CN); Wei Zhou, Hangzhou, Zhejiang (CN); Hangbin Miao, Hangzhou (CN); Guannv Wang, Hangzhou, Zhejiang (CN)

(73) Assignee: Zhejiang Shimai Pharmaceutical Co., Ltd., Hangzhou, Zhejiang (CN)

(21) Appl. No.: 18/574,000

(22) PCT Filed: May 17, 2022
(86) PCT No.: PCT/CN2022/093348

§ 371 Date: Dec. 22, 2023

(30) Foreign Application Priority Data
Jul. 23, 2021 (WO) PCT/CN2021/108155

(51) Int. Cl. C07K 16/46; A61K 39/00; A61P 35/00

(52) U.S. Cl. CPC C07K 16/468; A61P 35/00; A61K 2039/505; C07K 2317/31; C07K 2317/565

(57) ABSTRACT

Disclosed herein are monoclonal antibodies against ROR1, bispecific antibodies against ROR1 and CD3, nucleic acids comprising the antibodies, vectors comprising the nucleic acids, and host cell comprising the nucleic acids or the vectors. Also disclosed are pharmaceutical compositions and antibody-drug conjugates comprising the antibodies, and therapeutic methods for using the antibodies.

This international patent application claims the benefit of international patent application No.: PCT/CN2021/108155 filed on Jul. 23, 2021, the entire content of which is incorporated by reference for all purpose.

### FIELD OF THE INVENTION

The present invention is directed to antibodies against ROR1, and uses of such antibodies, in particular their use in the treatment of cancers.

### BACKGROUND OF THE INVENTION

ROR1 (receptor tyrosine kinase-like orphan receptor 1) is an evolutionarily conserved, type I membrane protein and is widely expressed in embryonic development and multiple human cancers. ROR1 has a cytoplasmic domain consisting of a tyrosine-kinase like domain, two serine/threonine-rich domains and a proline-rich domain (PRD); a transmembrane domain; and an extracellular domain consisting of an Ig-like domain, a frizzled domain and a kringle domain.

ROR1 serves as a Wnt5a receptor to induce noncanonical Wnt signaling, leading to enhanced leukemia cell migration and proliferation. It has been reported that ROR1 activates RhoA in chronic lymphocytic leukemia (CLL) cells to enhance migration. Wnt5a also induces HS1 (hematopoietic-lineage-specific protein 1) to undergo tyrosine phosphorylation and recruitment to the proline-rich domain of ROR1. Prior studies have reported that Wnt5a also induces ROR1/ROR2 hetero-oligomerization to recruit guanine exchange factors (GEFs) that activate Rho GTPases, which enhances leukemia chemotaxis and proliferation.

ROR1 expression attenuates during fetal development and, with few exceptions, becomes negligible on most postpartum tissues. In contrast, ROR1 is expressed by multiple human cancers, particularly those that are less differentiated, and is associated with early relapse after therapy or metastasis. Studies using flow cytometry demonstrated cell surface expression of ROR1 in multiple types of cancers including B-CLL, mantle cell lymphoma (MCL), and a subset of B-cell acute lymphoblastic leukemia (ALL). Expression of ROR1 enhances tumor cell growth and survival, and promotes epithelial-mesenchymal transition and metastasis of tumors. High ROR1 expression is correlated with shorter overall and metastasis-free survival in triple-negative breast cancer, lung adenocarcinoma, ovarian cancer, as well as other types of cancers.

Due to its expression pattern and function in tumor progression, ROR1 has become a compromising target for tumor therapy. There exists a need in the art for developing antigen bind proteins to ROR1, particularly those bind to ROR1 with high affinity and inhibitory activity.

### SUMMARY OF THE INVENTION

The present disclosure provides novel antibodies binding to ROR1 or antigen binding fragments thereof, which can be in a form of a monoclonal antibody or bispecific antibody, such as a bispecific T-cell engager (BITE). The antibodies disclosed herein are capable of binding to human ROR1, especially to the extracellular domain of human ROR1, with a high affinity, and mediating killing of effector cells against target cells expressing ROR1 (such as various cancer cells).

In an aspect, the present disclosure provides an antibody specifically binding to ROR1, or an antigen binding fragment thereof, comprising a light chain variable region (VL) and a heavy chain variable region (VH), wherein (i) the VL comprises LCDRs 1-3 having the amino acid sequences as shown in SEQ ID NOs: 2-4 respectively, and the VH comprises HCDRs 1-3 having the amino acid sequences as shown in SEQ ID NOs: 7-9 respectively; or (ii) the VL comprises LCDRs 1-3 having the amino acid sequences as shown in SEQ ID NOs: 12-14 respectively, and the VH comprises HCDRs 1-3 having the amino acid sequences as shown in SEQ ID NOs: 17-19 respectively.

In some embodiments of the antibody or the antigen binding fragment thereof disclosed herein, (i) the VL comprises an amino acid sequence having at least 80%, at least 85%, at least 90%, at least 95%, at least 98%, at least 99%, or 100% sequence identity to SEQ ID NO: 1 and the VH comprises an amino acid sequence having at least 80%, at least 85%, at least 90%, at least 95%, at least 98%, at least 99%, or 100% sequence identity to SEQ ID NO: 6; or (ii) the VL comprises an amino acid sequence having at least 80%, at least 85%, at least 90%, at least 95%, at least 98%, at least 99%, or 100% sequence identity to SEQ ID NO: 11 and the VH comprises an amino acid sequence having at least 80%, at least 85%, at least 90%, at least 95%, at least 98%, at least 99%, or 100% sequence identity to SEQ ID NO: 16.

In some embodiments of the antibody or the antigen binding fragment thereof disclosed herein, the antibody comprises (i) a light chain comprising an amino acid sequence having at least 80%, at least 85%, at least 90%, at least 95%, at least 98%, at least 99%, or 100% sequence identity to SEQ ID NO: 5 and a heavy chain comprising an amino acid sequence having at least 80%, at least 85%, at least 90%, at least 95%, at least 98%, at least 99%, or 100% sequence identity to SEQ ID NO: 10; or (ii) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20.

In some embodiments of the antibody or the antigen binding fragment thereof disclosed herein, the antibody is of an isotype selected from the group consisting of IgG, IgA, IgM, IgE and IgD.

In some embodiments of the antibody or the antigen binding fragment thereof disclosed herein, the antibody is of a subtype selected from the group consisting of IgG1, IgG2, IgG3, and IgG4.

In some embodiments of the antibody or the antigen binding fragment thereof disclosed herein, the antigen binding fragment is selected from the group consisting of Fab, Fab′, F(ab′)₂, Fd, Fd′, Fv, scFv, ds-scFv and dAb.

In some embodiments of the antibody or the antigen binding fragment thereof disclosed herein, the antibody is a monoclonal antibody.

In some embodiments of the antibody or the antigen binding fragment thereof disclosed herein, the antibody is a bi-specific or a multi-specific antibody.

In some embodiments of the antibody or the antigen binding fragment thereof disclosed herein, the antibody is a bispecific antibody which further comprises a second antigen binding region binding to a second antigen.

In some embodiments of the antibody or the antigen binding fragment thereof disclosed herein, the second antigen is a tumor associated antigen or an immune cell antigen.

In some embodiments of the antibody or the antigen binding fragment thereof disclosed herein, the second antigen is a T-cell antigen.

In some embodiments of the antibody or the antigen binding fragment thereof disclosed herein, the T-cell antigen is selected from the group consisting of T cell receptor (TCR), CD3, CD4, CD8, CD16, CD25, CD28, CD44, CD62L, CD69, ICOS, 41-BB (CD137), and NKG2D or any combination thereof.

In some embodiments of the antibody or the antigen binding fragment thereof disclosed herein, the second antigen is CD3, and the second antigen binding region comprises a VL and a VH, wherein the VL comprises LCDRs 1-3 having the amino acid sequences as shown in SEQ ID NOs: 22-24 respectively, and the VH comprises HCDRs 1-3 having the amino acid sequences as shown in SEQ ID NOs: 27-29 respectively.

In some embodiments of the antibody or the antigen binding fragment thereof disclosed herein, the second antigen binding region comprises a VL comprising an amino acid sequence having at least 80%, at least 85%, at least 90%, at least 95%, at least 98%, at least 99%, or 100% sequence identity to SEQ ID NO: 21 and a VH comprising an amino acid sequence having at least 80%, at least 85%, at least 90%, at least 95%, at least 98%, at least 99%, or 100% sequence identity to SEQ ID NO: 26.

In some embodiments of the antibody or the antigen binding fragment thereof disclosed herein, the VL of the second antigen binding region is linked to the C-terminal of the VL of the antibody specifically binding to ROR-1, and the VH of the second antigen binding region is linked to the C-terminal of the VH of the antibody specifically binding to ROR-1.

In some embodiments of the antibody or the antigen binding fragment thereof disclosed herein, the VL of the second antigen binding region is linked to the VL of the antibody specifically binding to ROR-1 via a linker having the amino acid sequence as shown in SEQ ID NO: 33, and the VH of the second antigen binding region is linked to the VH of the antibody specifically binding to ROR-1 via a linker having the amino acid sequence as shown in SEQ ID NO: 34.

In some embodiments of the antibody or the antigen binding fragment thereof disclosed herein, the bispecific antibody comprises (i) a light chain comprising an amino acid sequence having at least 80%, at least 85%, at least 90%, at least 95%, at least 98%, at least 99%, or 100% sequence identity to SEQ ID NO: 25 and a heavy chain comprising an amino acid sequence having at least 80%, at least 85%, at least 90%, at least 95%, at least 98%, at least 99%, or 100% sequence identity to SEQ ID NO: 30; or (ii) a light chain comprising an amino acid sequence having at least 80%, at least 85%, at least 90%, at least 95%, at least 98%, at least 99%, or 100% sequence identity to SEQ ID NO: 31 and a heavy chain comprising an amino acid sequence having at least 80%, at least 85%, at least 90%, at least 95%, at least 98%, at least 99%, or 100% sequence identity to SEQ ID NO: 32.

In some embodiments of the antibody or the antigen binding fragment thereof disclosed herein, the bispecific antibody is a bispecific T-cell engager (BiTE).

In another aspect, the present disclosure provides a bispecific antibody or an antigen binding fragment thereof, comprising a first antigen binding region binding to ROR1 comprising a VL and a VH and a second antigen binding region binding to CD3 comprising a VL and a VH, wherein (i) the VL of the first antigen binding region comprises LCDRs 1-3 having the amino acid sequences as shown in SEQ ID NOs: 2-4 respectively, and the VH of the first antigen binding region comprises HCDRs 1-3 having the amino acid sequences as shown in SEQ ID NOs: 7-9 respectively; or (ii) the VL of the first antigen binding region comprises LCDRs 1-3 having the amino acid sequences as shown in SEQ ID NOs: 12-14 respectively, and the VH of the first antigen binding region comprises HCDRs 1-3 having the amino acid sequences as shown in SEQ ID NOs: 17-19 respectively; and wherein the VL of the second antigen binding region comprises LCDRs 1-3 having the amino acid sequences as shown in SEQ ID NOs: 22-24 respectively, and the VH of the second antigen binding region comprises HCDRs 1-3 having the amino acid sequences as shown in SEQ ID NOs: 27-29 respectively.

In some embodiments of the bispecific antibody or the antigen binding fragment thereof disclosed herein, (i) the VL of the first antigen binding region comprises an amino acid sequence having at least 80%, at least 85%, at least 90%, at least 95%, at least 98%, at least 99%, or 100% sequence identity to SEQ ID NO: 1 and the VH of the first antigen binding region comprises an amino acid sequence having at least 80%, at least 85%, at least 90%, at least 95%, at least 98%, at least 99%, or 100% sequence identity to SEQ ID NO: 6; or (ii) the VL of the first antigen binding region comprises an amino acid sequence having at least 80%, at least 85%, at least 90%, at least 95%, at least 98%, at least 99%, or 100% sequence identity to SEQ ID NO: 11 and the VH of the first antigen binding region comprises an amino acid sequence having at least 80%, at least 85%, at least 90%, at least 95%, at least 98%, at least 99%, or 100% sequence identity to SEQ ID NO: 16; and the VL of the second antigen binding region comprises an amino acid sequence having at least 80%, at least 85%, at least 90%, at least 95%, at least 98%, at least 99%, or 100% sequence identity to SEQ ID NO: 21 and the VH of the second antigen binding region comprises an amino acid sequence having at least 80%, at least 85%, at least 90%, at least 95%, at least 98%, at least 99%, or 100% sequence identity to SEQ ID NO: 26.

In some embodiments of the bispecific antibody or the antigen binding fragment thereof disclosed herein, the VL of the second antigen binding region is linked to the C-terminal of the VL of the first antigen binding region, and the VH of the second antigen binding region is linked to the C-terminal of the VH of the first antigen binding region.

In some embodiments of the bispecific antibody or the antigen binding fragment thereof disclosed herein, the VL of the second antigen binding region is linked to the VL of the first antigen binding region via a linker having the amino acid sequence as shown in SEQ ID NO: 33, and the VH of the second antigen binding region is linked to the VH of the first antigen binding region via a linker having the amino acid sequence as shown in SEQ ID NO: 34.

In some embodiments of the bispecific antibody or the antigen binding fragment thereof disclosed herein, the bispecific antibody comprises (i) a light chain comprising an amino acid sequence having at least 80%, at least 85%, at least 90%, at least 95%, at least 98%, at least 99%, or 100% sequence identity to SEQ ID NO: 25 and a heavy chain comprising an amino acid sequence having at least 80%, at least 85%, at least 90%, at least 95%, at least 98%, at least 99%, or 100% sequence identity to SEQ ID NO: 30; or (ii) a light chain comprising an amino acid sequence having at least 80%, at least 85%, at least 90%, at least 95%, at least 98%, at least 99%, or 100% sequence identity to SEQ ID NO: 31 and a heavy chain comprising an amino acid sequence having at least 80%, at least 85%, at least 90%, at least 95%, at least 98%, at least 99%, or 100% sequence identity to SEQ ID NO: 32.

In some embodiments of the bispecific antibody or the antigen binding fragment thereof disclosed herein, the bispecific antibody is a bispecific T-cell engager (BiTE).

In yet another aspect, the present disclosure provides a nucleic acid comprising a nucleotide sequence encoding the antibody or the antigen binding fragment thereof disclosed herein or the bispecific antibody or the antigen binding fragment thereof disclosed herein.

In still another aspect, the present disclosure provides a vector comprising the nucleic acid disclosed herein.

In another aspect, the present disclosure provides a host cell comprising the nucleic acid disclosed herein or the vector disclosed herein.

In yet another aspect, the present disclosure provides a pharmaceutical composition comprising (i) the antibody or the antigen binding fragment thereof disclosed herein, or the bispecific antibody or the antigen binding fragment thereof disclosed herein; and (ii) a pharmaceutically acceptable carrier or adjuvant.

In still another aspect, the present disclosure provides an antibody-drug conjugate, comprising the antibody or the antigen binding fragment thereof disclosed herein, or the bispecific antibody or the antigen binding fragment thereof disclosed herein.

In another aspect, the present disclosure provides a method of treating a cancer in a subject, comprising administering to the subject an effective amount of the antibody or the antigen binding fragment thereof disclosed herein, the bispecific antibody or the antigen binding fragment thereof disclosed herein, the pharmaceutical composition disclosed herein, or the antibody-drug conjugate disclosed herein.

In some embodiments of the method disclosed herein, the cancer is selected from the group consisting of breast cancer, lung cancer, ovarian cancer, colon cancer, liver cancer, esophageal cancer, pancreatic cancer, bladder cancer, prostate cancer, colorectal cancer, uterine cancer, cervical cancer, brain cancer, cervical cancer, gastric cancer, cholangiocarcinoma, chondrosarcoma, kidney cancer, thyroid cancer, skin cancer, lymphoma, myeloma, and leukemia, preferably selected from the group consisting of chronic lymphocytic leukemia (CLL), mantle cell lymphoma (MCL), B-cell acute lymphoblastic leukemia, T-cell acute lymphoblastic leukemia, Burkitt lymphoma, multiple myeloma, lung adenocarcinoma, non-small cell lung cancer (NSCLC), human esophageal squamous cell carcinoma, colonic adenocarcinoma, breast cancer, pancreatic cancer, bladder cancer, colorectal cancer, liver cancer, and ovarian cancer.

In some embodiments of the method disclosed herein, the method further comprises administering to the subject a second therapeutic agent.

In some embodiments of the method disclosed herein, the second therapeutic agent is selected from an antibody, a chemotherapeutic agent and a small molecule drug.

In some embodiments of the method disclosed herein, the second therapeutic agent is selected from a Bruton's tyrosine kinase (BTK) inhibitor, a PI3K inhibitor, a HDAC inhibitor, a PD-1/PD-L1 inhibitor, a LAG3 inhibitor, and glucocorticoid.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows binding of 2H3 and 3A3 Fabs against recombinant human ROR1.

FIG. 2A shows binding of anti-ROR1 monoclonal antibody 2H3 (2H3 mAb) against full extracellular domain or individual extracellular Ig-like domain, frizzled domain and kringle domain of recombinant human ROR1 as measured by ELISA.

FIG. 2B shows binding of anti-ROR1 monoclonal antibody 3A3 (3A3 mAb) against full extracellular domain or individual extracellular Ig-like domain, frizzled domain and kringle domain of recombinant human ROR1 as measured by ELISA.

FIG. 3 shows binding of 2H3 and 3A3 mAbs to cell surface-associated ROR1 in cancer cell lines MDAMB231, H1975, JEKO-1, KYSE30, PANC-1 and H460, as measured by flow cytometry. Color code, purple: negative control; green: 2H3 mAb; red: 3A3 mAb. Antibody concentration used for assay is 10 μg/ml.

FIG. 4A shows binding of bispecific antibodies targeting ROR1 and CD3 (2H3 and 3A3 HBiTEs) against recombinant human CD3 as measured by ELISA.

FIG. 4B shows binding of 2H3 and 3A3 HBiTEs against recombinant human ROR1 as measured by ELISA.

FIG. 5A shows binding of 2H3 HBiTE (ROR1-2H3-HB) to ROR1 positive cell line JEKO-1 and CD3 positive cell line Jurkat as measured by flow cytometry.

FIG. 5B shows binding of 3A3 HBiTE (ROR1-3A3-HB) to ROR1 positive cell line JEKO-1 and CD3 positive cell line Jurkat as measured by flow cytometry.

FIG. 6 shows killing activity of 2H3 and 3A3 bispecific antibodies against JEKO-1 cells in the presence of human PBMC. PBMC cells were added at a ratio of 1:5 of target cells (JEKO-1) to effector cells (PBMC).

FIG. 7 shows killing activity of 2H3 and 3A3 bispecific antibodies against MDA-MB-231 cells in the presence of human PBMC. The ratio of target cells (MDA-MB-231) to effector cells (PBMC) is 1:5.

FIG. 8 shows killing activity of 2H3 and 3A3 bispecific antibodies against SK-HEP-1 cells in the presence of human PBMC. The ratio of target cells (SK-HEP-1) to effector cells (PBMC) is 1:5.

FIG. 9 shows killing activity of 2H3 and 3A3 bispecific antibodies against PANC-1 cells in the presence of human PBMC. The ratio of target cells (PANC-1) to effector cells (PBMC) is 1:5.

FIG. 10A shows ADCC killing of 2H3 Mab and 3A3 Mab against HT29 cells in the presence of NK cells.

FIG. 10B shows images of ADCC killing of 2H3 Mab and 3A3 Mab against HT29 cells in the presence of NK cells.

FIG. 11A shows inhibition of tumor volume by 2H3 bispecific antibody in mice model. Saline solution is used as negative control.

FIG. 11B shows inhibition of tumor weight by 2H3 bispecific antibody in mice model. Saline solution is used as negative control.

FIG. 12A shows inhibition of tumor volume by 3A3 bispecific antibody in mice model. Saline solution is used as negative control.

FIG. 12B shows inhibition of tumor weight by 3A3 bispecific antibody in mice model. Saline solution is used as negative control.

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland; Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

Unless indicated or defined otherwise, the term “comprise”, and variations such as “comprises” and “comprising”, should be understood to imply the inclusion of a stated elements or step or group of elements or steps but not the exclusion of any other element or step or group of elements or steps.

In an aspect, the present disclosure provides an antibody specifically binding to ROR1, or an antigen binding fragment thereof, comprising a light chain variable region (VL) and a heavy chain variable region (VH), wherein (i) the VL comprises LCDRs 1-3 having the amino acid sequences as shown in SEQ ID NOs: 2-4 respectively, and the VH comprises HCDRs 1-3 having the amino acid sequences as shown in SEQ ID NOs: 7-9 respectively; or (ii) the VL comprises LCDRs 1-3 having the amino acid sequences as shown in SEQ ID NOs: 12-14 respectively, and the VH comprises HCDRs 1-3 having the amino acid sequences as shown in SEQ ID NOs: 17-19 respectively.

As used herein, the term “antibody” refers to an immunoglobulin molecule which has the ability to specifically bind to a specific antigen. An antibody often comprises a variable region and a constant region in each of a heavy chain and a light chain. The variable regions of the heavy and light chains of antibodies contain a binding domain that interacts with an antigen. The constant regions of antibodies may mediate the binding of the immunoglobulin to host tissues or factors, including various cells of the immune system (such as effector cells) and components of the complement system such as C1q, the first component in the classical pathway of complement activation. Accordingly, most antibodies have a heavy chain variable region (VH) and a light chain variable region (VL) that together form the portion of the antibody that binds to the antigen.

A “light chain variable region” (VL) or “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering system) in which corresponding residues between different heavy chains or between different light chains are assigned the same number. The present disclosure can use CDRs defined according to any of these numbering systems, although preferred embodiments use Kabat or Chothia defined CDRs.

When CDR sequences are defined according to Kabat numbering system, the VL of the antibody disclosed herein comprises LCDR1, LCDR2 and LCDR3 having the amino acid sequences as shown in SEQ ID NO: 2 (RASQSVSSYLA), SEQ ID NO: 3 (DASNRAT) and SEQ ID NO: 4 (QQRSNWPLT) respectively, and the VH of the antibody disclosed herein comprises HCDR1, HCDR2 and HCDR3 having the amino acid sequences as shown in SEQ ID NO: 7 (GYTFTYR), SEQ ID NO: 8 (TPFNGN) and SEQ ID NO: 9 (SGPRGDYVLDY) respectively; or
the VL of the antibody disclosed herein comprises LCDR1, LCDR2 and LCDR3 having the amino acid sequences as shown in SEQ ID NO: 12 (RSSQSLLQSNGYNYVE), SEQ ID NO: 13 (LGSYRAS) and SEQ ID NO: 14 (MQGTHWPLFT) respectively, and the VH of the antibody disclosed herein comprises HCDR1, HCDR2 and HCDR3 having the amino acid sequences as shown in SEQ ID NO: 17 (GFTFSSY), SEQ ID NO: 18 (SYDGSN) and SEQ ID NO: 19 (DLDYSLWFDP) respectively.

The term “antibody” as used herein should be understood in its broadest meaning, and includes monoclonal antibodies (including full-length monoclonal antibodies), polyclonal antibodies, antibody fragments, and multispecific antibodies containing at least two different antigen binding regions (e.g., bispecific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

As used herein, the term “binding” or “specifically binding” refers to a non-random binding reaction between two molecules, such as between an antibody and its target antigen. In certain embodiments, an antibody specifically binding to a certain antigen refers to an antibody binding to the antigen with an affinity corresponding to a KD of less than about 10⁻⁵ M, for example, less than about 10⁻⁶ M, 10⁻⁷ M, 10⁻⁸ M, 10⁻⁹ M, or 10⁻¹⁰ M or less. As used herein, “KD” refers to the dissociation equilibrium constant of a particular antibody-antigen interaction, and is used to describe binding affinity between an antibody and an antigen. The smaller the KD, the higher the binding affinity between the antibody and the antigen.

As used herein, the term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

In some embodiments of the antibody or the antigen binding fragment thereof disclosed herein, (i) the VL comprises an amino acid sequence having at least 80%, at least 85%, at least 90%, at least 95%, at least 98%, at least 99%, or 100% sequence identity to SEQ ID NO: 1 and the VH comprises an amino acid sequence having at least 80%, at least 85%, at least 90%, at least 95%, at least 98%, at least 99%, or 100% sequence identity to SEQ ID NO: 6; or (ii) the VL comprises an amino acid sequence having at least 80%, at least 85%, at least 90%, at least 95%, at least 98%, at least 99%, or 100% sequence identity to SEQ ID NO: 11 and the VH comprises an amino acid sequence having at least 80%, at least 85%, at least 90%, at least 95%, at least 98%, at least 99%, or 100% sequence identity to SEQ ID NO: 16.

Percent (%) of “sequence identity” herein refers to the extent to which two sequences (amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

In some embodiments of the antibody or the antigen binding fragment thereof disclosed herein, the antibody comprises (i) a light chain comprising an amino acid sequence having at least 80%, at least 85%, at least 90%, at least 95%, at least 98%, at least 99%, or 100% sequence identity to SEQ ID NO: 5 and a heavy chain comprising an amino acid sequence having at least 80%, at least 85%, at least 90%, at least 95%, at least 98%, at least 99%, or 100% sequence identity to SEQ ID NO: 10; or (ii) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20.

Based on the amino acid sequence of heavy chain constant regions of the antibody, a immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (λ) chain or a kappa (κ) chain, based on the amino acid sequence of the light chain. The antibodies disclosed herein can be of any classes or subtypes above.

In some embodiments of the antibody or the antigen binding fragment thereof disclosed herein, the antibody is of an isotype selected from the group consisting of IgG, IgA, IgM, IgE and IgD. In some embodiments, the antibody is of a subtype selected from the group consisting of IgG1, IgG2, IgG3, and IgG4.

As used herein, the term “antigen binding fragment” includes but is not limited to: a Fab fragment having VL, CL, VH, and CH1 domains; a Fab′ fragment having one or more cysteine residues at the C-terminus of CH1 domain of the Fab fragments; a Fd fragment having VH and CH1 domains; a Fd′ fragment having VH and CH1 domains and one or more cysteine residues at the C-terminus of the CH1 domain; a Fv fragment and scFv, which have VL and VH domains in a single arm of an antibody; a dAb fragment consisting of VH domains or VL domains; isolated CDR regions; a F(ab′)₂ fragment, bivalent fragments comprising two Fab′ fragments linked by a disulfide bridge at the hinge region; a “linear antibody” comprising a pair of tandem Fd segments (VH-CH1-VH-CH1), which forms an antigen binding region together with a complementary light chain polypeptide; and a modified version of any of the foregoing fragments, which retains antigen binding activity.

In some embodiments of the antibody or the antigen binding fragment thereof disclosed herein, the antigen binding fragment is selected from the group consisting of Fab, Fab′, F(ab′)₂, Fd, Fd′, Fv, scFv, ds-scFv and dAb.

In some embodiments of the antibody or the antigen binding fragment thereof disclosed herein, the antibody is a monoclonal antibody.

As used herein, the term “monoclonal antibody” refers to an antibody obtained from a substantially homogeneous antibody population. That is, each antibodies constituting the population are the same, except for possible naturally occurring mutations in small amount. Monoclonal antibodies are highly specific and are directed against a single antigen. The term “monoclonal antibody” herein is not limited to antibodies produced by hybridoma technology, and should not be interpreted as requiring production of antibodies by any specific method.

In some embodiments of the antibody or the antigen binding fragment thereof disclosed herein, the antibody is a bi-specific or a multi-specific antibody. In some embodiments, the antibody is a bispecific antibody which further comprises a second antigen binding region binding to a second antigen.

In some embodiments of the antibody or the antigen binding fragment thereof disclosed herein, the second antigen is a tumor associated antigen or an immune cell antigen.

Many tumor associated antigens associated with specific cancers have been identified in the art. As used herein, the term “tumor associated antigen” refers to an antigen that is differentially expressed in cancer cells compared to normal cells, and therefore can be used to target cancer cells. In some embodiments, tumor-associated antigens are antigens that can potentially stimulate an obvious tumor-specific immune response. Some of these antigens are encoded by normal cells, but not necessarily expressed by normal cells. These antigens can be characterized as those that are usually silent (i.e., not expressed) in normal cells, those that are expressed only during certain stages of differentiation, and those that are expressed over time, such as embryonic and fetal antigens. Other cancer antigens are encoded by mutant cell genes such as oncogenes (e.g. activated ras oncogene), suppressor genes (e.g. mutant p53), and fusion proteins produced by internal deletions or chromosomal translocations. Other cancer antigens can be encoded by viral genes, such as those carried on RNA and DNA tumor viruses. Many other tumor associated antigens and antibodies against them are known and/or commercially available, and can also be produced by those skilled in the art.

In some embodiments of the antibody or the antigen binding fragment thereof disclosed herein, the second antigen is a T-cell antigen. In some embodiments, the T-cell antigen is selected from the group consisting of T cell receptor (TCR), CD3, CD4, CD8, CD16, CD25, CD28, CD44, CD62L, CD69, ICOS, 41-BB (CD137), and NKG2D or any combination thereof. In some embodiments, the T-cell antigen is CD3, and the second antigen binding region binds to any of γ chain, δ chain, ε chain, ζ chain and η chain of CD3.

In some embodiments of the antibody or the antigen binding fragment thereof disclosed herein, the second antigen is CD3, and the second antigen binding region comprises a VL and a VH, wherein the VL comprises LCDRs 1-3 having the amino acid sequences as shown in SEQ ID NOs: 22-24 respectively, and the VH comprises HCDRs 1-3 having the amino acid sequences as shown in SEQ ID NOs: 27-29 respectively.

In some embodiments, CDR sequences are defined according to Kabat numbering system. When using Kabat defined CDR sequences, the VL of the second antigen binding region disclosed herein comprises LCDR1, LCDR2 and LCDR3 having the amino acid sequences as shown in SEQ ID NO: 22 (RSSTGAVTTSNYAN), SEQ ID NO: 23 (GANKRAP) and SEQ ID NO: 24 (ALWYSNLWV) respectively, and the VH of the second antigen binding region disclosed herein comprises HCDR1, HCDR2 and HCDR3 having the amino acid sequences as shown in SEQ ID NO: 27 (GFTFNTY), SEQ ID NO: 28 (RSKYNNYA) and SEQ ID NO: 29 (HGNFGSSYVSYFAY) respectively.

In some embodiments of the antibody or the antigen binding fragment thereof disclosed herein, the second antigen binding region comprises a VL comprising an amino acid sequence having at least 80%, at least 85%, at least 90%, at least 95%, at least 98%, at least 99%, or 100% sequence identity to SEQ ID NO: 21 and a VH comprising an amino acid sequence having at least 80%, at least 85%, at least 90%, at least 95%, at least 98%, at least 99%, or 100% sequence identity to SEQ ID NO: 26.

In some embodiments of the antibody or the antigen binding fragment thereof disclosed herein, the VL of the second antigen binding region is linked to the C-terminal of the VL of the antibody specifically binding to ROR-1, and the VH of the second antigen binding region is linked to the C-terminal of the VH of the antibody specifically binding to ROR-1.

In some embodiments of the antibody or the antigen binding fragment thereof disclosed herein, the VL of the second antigen binding region is linked to the VL of the antibody specifically binding to ROR-1 via a linker having the amino acid sequence as shown in SEQ ID NO: 33 (GGGGSGGGGSGGGGS), and the VH of the second antigen binding region is linked to the VH of the antibody specifically binding to ROR-1 via a linker having the amino acid sequence as shown in SEQ ID NO: 34 (GGGSSGGGGSGGGGS).

In some embodiments of the antibody or the antigen binding fragment thereof disclosed herein, the bispecific antibody comprises (i) a light chain comprising an amino acid sequence having at least 80%, at least 85%, at least 90%, at least 95%, at least 98%, at least 99%, or 100% sequence identity to SEQ ID NO: 25 and a heavy chain comprising an amino acid sequence having at least 80%, at least 85%, at least 90%, at least 95%, at least 98%, at least 99%, or 100% sequence identity to SEQ ID NO: 30; or (ii) a light chain comprising an amino acid sequence having at least 80%, at least 85%, at least 90%, at least 95%, at least 98%, at least 99%, or 100% sequence identity to SEQ ID NO: 31 and a heavy chain comprising an amino acid sequence having at least 80%, at least 85%, at least 90%, at least 95%, at least 98%, at least 99%, or 100% sequence identity to SEQ ID NO: 32.

In some embodiments of the antibody or the antigen binding fragment thereof disclosed herein, the bispecific antibody is a bispecific T-cell engager (BiTE).

As used herein, the term “bispecific T-cell engager” or “BiTE” refers to single polypeptide chain molecules that having two antigen-binding domains, one of which binds to a T-cell antigen and the second of which binds to an antigen present on the surface of a target (See, PCT Publication WO 05/061547; Bacuerle et al., 2008, Drugs of the Future 33: 137-147; Bargou, et al., 2008, Science 321:974-977, incorporated herein by reference in their entireties). Thus, the BiTEs of the disclosure have an antigen binding region that binds to ROR1 and a second antigen binding region that is directed towards a T-cell antigen.

In some embodiments of the antibody or the antigen binding fragment thereof disclosed herein, the bispecific antibody is in form of an HBiTE as described in PCT application No. PCT/US2018/016524 (which is incorporated herein by reference in its entirety). In the HBiTE, the light chain, from N-terminus to C-terminus, comprises an anti-target VL domain, an anti-CD3 VL-CL and a monomeric human IgG1 Fc (e.g., mFc7.2); and the heavy chain, from N-terminus to C-terminus, comprises an anti-target VH domain, an anti-CD3 VH-CH1 and a monomeric human IgG1 Fc (e.g., mFc7.2). Monomeric Fc7.2 contains two amino acid mutations (T366L and Y407H) capable of decreasing Fc homodimerization.

In another aspect, the present disclosure provides a bispecific antibody or an antigen binding fragment thereof, comprising a first antigen binding region binding to ROR1 comprising a VL and a VH and a second antigen binding region binding to CD3 comprising a VL and a VH, wherein (i) the VL of the first antigen binding region comprises LCDRs 1-3 having the amino acid sequences as shown in SEQ ID NOs: 2-4 respectively, and the VH of the first antigen binding region comprises HCDRs 1-3 having the amino acid sequences as shown in SEQ ID NOs: 7-9 respectively; or (ii) the VL of the first antigen binding region comprises LCDRs 1-3 having the amino acid sequences as shown in SEQ ID NOs: 12-14 respectively, and the VH of the first antigen binding region comprises HCDRs 1-3 having the amino acid sequences as shown in SEQ ID NOs: 17-19 respectively; and wherein the VL of the second antigen binding region comprises LCDRs 1-3 having the amino acid sequences as shown in SEQ ID NOs: 22-24 respectively, and the VH of the second antigen binding region comprises HCDRs 1-3 having the amino acid sequences as shown in SEQ ID NOs: 27-29 respectively.

In some embodiments of the bispecific antibody or the antigen binding fragment thereof disclosed herein, (i) the VL of the first antigen binding region comprises an amino acid sequence having at least 80%, at least 85%, at least 90%, at least 95%, at least 98%, at least 99%, or 100% sequence identity to SEQ ID NO: 1 and the VH of the first antigen binding region comprises an amino acid sequence having at least 80%, at least 85%, at least 90%, at least 95%, at least 98%, at least 99%, or 100% sequence identity to SEQ ID NO: 6; or (ii) the VL of the first antigen binding region comprises an amino acid sequence having at least 80%, at least 85%, at least 90%, at least 95%, at least 98%, at least 99%, or 100% sequence identity to SEQ ID NO: 11 and the VH of the first antigen binding region comprises an amino acid sequence having at least 80%, at least 85%, at least 90%, at least 95%, at least 98%, at least 99%, or 100% sequence identity to SEQ ID NO: 16; and the VL of the second antigen binding region comprises an amino acid sequence having at least 80%, at least 85%, at least 90%, at least 95%, at least 98%, at least 99%, or 100% sequence identity to SEQ ID NO: 21 and the VH of the second antigen binding region comprises an amino acid sequence having at least 80%, at least 85%, at least 90%, at least 95%, at least 98%, at least 99%, or 100% sequence identity to SEQ ID NO: 26.

In some embodiments of the bispecific antibody or the antigen binding fragment thereof disclosed herein, the VL of the second antigen binding region is linked to the C-terminal of the VL of the first antigen binding region, and the VH of the second antigen binding region is linked to the C-terminal of the VH of the first antigen binding region.

In some embodiments of the bispecific antibody or the antigen binding fragment thereof disclosed herein, the VL of the second antigen binding region is linked to the VL of the first antigen binding region via a linker having the amino acid sequence as shown in SEQ ID NO: 33 (GGGGSGGGGSGGGGS), and the VH of the second antigen binding region is linked to the VH of the first antigen binding region via a linker having the amino acid sequence as shown in SEQ ID NO: 34 (GGGSSGGGGSGGGGS).

In some embodiments of the bispecific antibody or the antigen binding fragment thereof disclosed herein, the bispecific antibody comprises (i) a light chain comprising an amino acid sequence having at least 80%, at least 85%, at least 90%, at least 95%, at least 98%, at least 99%, or 100% sequence identity to SEQ ID NO: 25 and a heavy chain comprising an amino acid sequence having at least 80%, at least 85%, at least 90%, at least 95%, at least 98%, at least 99%, or 100% sequence identity to SEQ ID NO: 30; or (ii) a light chain comprising an amino acid sequence having at least 80%, at least 85%, at least 90%, at least 95%, at least 98%, at least 99%, or 100% sequence identity to SEQ ID NO: 31 and a heavy chain comprising an amino acid sequence having at least 80%, at least 85%, at least 90%, at least 95%, at least 98%, at least 99%, or 100% sequence identity to SEQ ID NO: 32.

In some embodiments of the bispecific antibody or the antigen binding fragment thereof disclosed herein, the bispecific antibody is a bispecific T-cell engager (BiTE).

In yet another aspect, the present disclosure provides a nucleic acid comprising a nucleotide sequence encoding the antibody or the antigen binding fragment thereof disclosed herein or the bispecific antibody or the antigen binding fragment thereof disclosed herein.

In still another aspect, the present disclosure provides a vector comprising the nucleic acid disclosed herein.

As used herein, the term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In some embodiments, vectors include but are not limited to: (1) plasmids; (2) phagemids; (3) cosmids; (4) artificial chromosomes, such as yeast artificial chromosomes, bacterial artificial chromosomes or artificial chromosomes derived from P1; (5) phage, such as lambda phage or M13 phage; and (6) animal viruses, such as retrovirus, adenovirus, adeno-associated virus, sporangia virus, poxvirus, baculovirus.

In another aspect, the present disclosure provides a host cell comprising the nucleic acid disclosed herein or the vector disclosed herein.

As used herein, the term “host cell” refers to a cell into which an expression vector has been introduced. In some embodiments, host cells include, for example, CHO cells, such as CHOS cells and CHO-K1 cells, or HEK293 cells, such as HEK293A, HEK293T and HEK293F.

In yet another aspect, the present disclosure provides a pharmaceutical composition comprising (i) the antibody or the antigen binding fragment thereof disclosed herein, or the bispecific antibody or the antigen binding fragment thereof disclosed herein; and (ii) a pharmaceutically acceptable carrier or adjuvant.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

In some embodiments, the carrier or adjuvant for use with the composition disclosed herein includes but is not limited to maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In still another aspect, the present disclosure provides an antibody-drug conjugate, comprising the antibody or the antigen binding fragment thereof disclosed herein, or the bispecific antibody or the antigen binding fragment thereof disclosed herein. In some embodiments, the drug is toxic chemotherapeutic drugs such as maytansine, geldanamycin, tubulin inhibitors such as tubulin binding agents (e.g., auristatins), or minor groove binding agents such as calicheamicin.

In another aspect, the present disclosure provides a method of treating a cancer in a subject, comprising administering to the subject an effective amount of the antibody or the antigen binding fragment thereof disclosed herein, the bispecific antibody or the antigen binding fragment thereof disclosed herein, the pharmaceutical composition disclosed herein, or the antibody-drug conjugate disclosed herein.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g. cancers). The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment, or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human.

In some embodiments of the method disclosed herein, the cancer is a cancer associated with the expression of ROR1. In some embodiments, the cancer is selected from the group consisting of breast cancer, lung cancer, ovarian cancer, colon cancer, liver cancer, esophageal cancer, pancreatic cancer, bladder cancer, prostate cancer, colorectal cancer, uterine cancer, cervical cancer, brain cancer, cervical cancer, gastric cancer, cholangiocarcinoma, chondrosarcoma, kidney cancer, thyroid cancer, skin cancer, lymphoma, myeloma, and leukemia, preferably selected from the group consisting of chronic lymphocytic leukemia (CLL), mantle cell lymphoma (MCL), B-cell acute lymphoblastic leukemia, T-cell acute lymphoblastic leukemia, Burkitt lymphoma, multiple myeloma, lung adenocarcinoma, non-small cell lung cancer (NSCLC), human esophageal squamous cell carcinoma, colonic adenocarcinoma, breast cancer, pancreatic cancer, bladder cancer, colorectal cancer, liver cancer, and ovarian cancer.

In some embodiments of the method disclosed herein, the method further comprises administering to the subject a second therapeutic agent. In some embodiments, the second therapeutic agent is selected from an antibody, a chemotherapeutic agent and a small molecule drug.

In some embodiments, the therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some embodiments of the method disclosed herein, the second therapeutic agent is selected from a Bruton's tyrosine kinase (BTK) inhibitor, a PI3K inhibitor, a HDAC inhibitor, a PD-1/PD-L1 inhibitor, a LAG3 inhibitor, and glucocorticoid.

In another aspect, the present disclosure provides use of the antibody or the antigen binding fragment thereof disclosed herein, the bispecific antibody or the antigen binding fragment thereof disclosed herein, the pharmaceutical composition disclosed herein, or the antibody-drug conjugate disclosed herein in the manufacture of a medicament for treating a cancer in a subject. In some embodiments, the cancer is a cancer associated with the expression of ROR1, preferably selected from the group consisting of chronic lymphocytic leukemia (CLL), mantle cell lymphoma (MCL), B-cell acute lymphoblastic leukemia, T-cell acute lymphoblastic leukemia, Burkitt lymphoma, multiple myeloma, lung adenocarcinoma, non-small cell lung cancer (NSCLC), human esophageal squamous cell carcinoma, colonic adenocarcinoma, breast cancer, pancreatic cancer, bladder cancer, colorectal cancer, liver cancer, and ovarian cancer.

In yet another aspect, the present disclosure provides the antibody or the antigen binding fragment thereof disclosed herein, the bispecific antibody or the antigen binding fragment thereof disclosed herein, the pharmaceutical composition disclosed herein, or the antibody-drug conjugate disclosed herein for use in treating a cancer in a subject. In some embodiments, the cancer is a cancer associated with the expression of ROR1, preferably selected from the group consisting of chronic lymphocytic leukemia (CLL), mantle cell lymphoma (MCL), B-cell acute lymphoblastic leukemia, T-cell acute lymphoblastic leukemia, Burkitt lymphoma, multiple myeloma, lung adenocarcinoma, non-small cell lung cancer (NSCLC), human esophageal squamous cell carcinoma, colonic adenocarcinoma, breast cancer, pancreatic cancer, bladder cancer, colorectal cancer, liver cancer, and ovarian cancer.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

HT29 cells (human colorectal cancer cell line) and BJAB cells (human Burkitt-like lymphoma cell line) were purchased from ATCC. MDA-MB-231 cancer cells (human breast cancer cell line) were donated by Cancer Institute of the Second Affiliated Hospital of Zhejiang University (School of Medicine). Other cells including COLO205 (human colorectal cancer cell line), H460 (human non-small cell lung cancer cell line), KYSE30 (human esophageal squamous cell carcinoma cell line), LS174T (human colonic adenocarcinoma cell line), H1975 (human non-small cell lung cancer cell line), PANC-1 (human pancreatic cancer cell line), JEKO-1 (human mantle cell lymphoma cell line), RPMIH8226 (human multiple myeloma cell line), 5637 (human bladder cancer cell line), and Jurkat cancer cells (T-cell acute lymphoblastic leukemia cell line) were purchased from National Collection of Authenticated Cell Cultures.

Biotinylated human ROR1 protein, human ROR1 protein, human ROR1 (165-305, Frizzled domain) protein, human ROR1 (39-151, Ig-like domain) protein, human/Cynomolgus/Rhesus macaque ROR1 (308-395, Kringle domain) protein, and human ROR2/NTRKR2 protein were purchased from ACROBiosystems. Mouse ROR1 protein was purchased from Sino Biological.

Anti-human IgG (γ-chain specific)-R-PE antibody, anti-human IgG (Fc-specific)-peroxidase antibody and monoclonal anti-Flag® M2-peroxidase were purchased from of Sigma. M13KO7 helper phage was purchased from of New England Biolabs. Dynabeads™ Myone™ Streptavidin T1 was purchased from ThermoFisher Scientific. PE anti-His tag antibody was purchased from BioLegend. M13 bacteriophage antibody (HRP) was purchased from Sino Biological.

### Example 1. Panning and Screening of a Phage-Display Naive Human Fab Library for Identification of ROR1 Antibodies

A large (size, 10¹¹) phage-display naive human Fab library with peripheral blood B cells from about 30 healthy individuals was used for selection of antibodies against recombinant human ROR1 conjugated to magnetic beads (Dynabeads™ Myone™ Streptavidin T1; ThermoFisher Scientific) as described previously (Zhu et al., J Virol 2006, 80:891-899) with minor modification that 5, 1, 0.2 and 0.2 mg of antigen was used in the first, second, third and fourth round of panning, respectively. Clones that bound to the antigen were identified from the 4^{th} round of biopanning by using monoclonal phage ELISA. The 4^{th} round phage was subsequently used for specific binding identification. By soluble expression-based monoclonal enzyme-linked immunosorbent assay (SemELISA) and sequencing analysis, two specific Fab clones, designated as 2H3 and 3A3, were identified. Both 2H3 and 3A3 Fabs have a κ light chain. The 3A3 light chain has a slightly longer CDR1 consisting of 16 amino acid residues with the Kabat system.

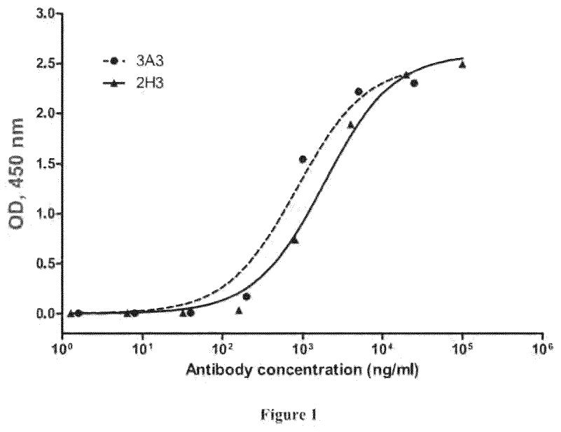

The hexahistidine-tagged Fabs 2H3 and 3A3 were expressed in E. coli strain HB2151 and purified from the soluble fraction of periplasm by using the Ni-NTA resin. Then ELISA was performed by using standard protocols to measure binding affinity to recombinant human ROR1 (full-length extracellular domain). Briefly, the recombinant human ROR1 (ACROBiosystems) was coated on Corning EIA/RIA high-binding 96-well plates (Corning Inc.) at 50 ng per well overnight at 4° C. and blocked with 3% nonfat milk in PBS (pH7.4). Fivefold serially diluted antibodies were added and incubated at room temperature for 2 h. The plates were washed with PBS containing 0.05% Tween 20. Bound antibodies were detected by HRP-conjugated anti-FLAG tag antibody (Sino Biological). The assay was developed at room temperature with TMB substrate (Solarbio) and OD value was measured at 450 nm with a microplate reader. The results showed that Fab clones 2H3 and 3A3 have high affinity with EC50 of approximately 16 nM and 36 nM, respectively (FIG. 1). This demonstrates that both Fab clones have high affinity to human ROR1, which enables development for therapeutic antibodies.

### Example 2. Construction and Initial Characterization of Anti-ROR1 Monoclonal Antibodies

Fab clones 2H3 and 3A3 having high affinity to human ROR1 were used to construct intact monoclonal antibodies 2H3 and 3A3. Briefly, the heavy chain Fd fragments of Fab clones 2H3 and 3A3 were fused to the N-terminus of human IgG1 Fc fragment, respectively. Both light chain and heavy chain were constructed into the vector pDin1 modified by the inventors for the expression of monoclonal antibodies, which comprises two molecular cloning sites. Construction and initial characterization of the two anti-ROR1 monoclonal antibodies were performed as follow.

### Cloning of ROR1 Monoclonal Antibodies

To generate constructs of anti-ROR1 monoclonal antibodies, following primers were used:

ROR1-2H3-LC-FP,
(SEQ ID NO: 35)
5′ AGATGCCAGATGTGAAATTGTGTTGAC 3′(sense);
ROR1-2H3-LC-RP,
(SEQ ID NO: 36)
5′ ATTTTGAGCTCTTAACACTCTCCCCTGTTGAAGCTCTTTGTGACGGG
CGAGGACAGGCCCTGATGGGT 3′(antisense);
ROR1-2H3-HC-FP,
(SEQ ID NO: 37)
5′ ACTACAGGTGTCCACTCCCAGGTGCAGCTGGTA 3′(sense);
ROR1-2H3-CH1-RP,
(SEQ ID NO: 38)
5′ ACAAGATTTGGGCTCAACTTTCTTGT 3′(antisense);
ROR1-2H3-FC-FP,
(SEQ ID NO: 39)
5′ AGTTGAGCCCAAATCTTGTGACAAAACTCACACA 3′(sense);
ROR1-2H3-FC-RP,
(SEQ ID NO: 40)
5′ ACGCGGATCCTTATTTACCCGGGGACAGGGA 3′
(antisense);
ROR1-3A3-LC-FP,
(SEQ ID NO: 41)
5′ AGATGCCAGATGTGATGTTGTGATGAC 3′(sense);
ROR1-3A3-HC-FP,
(SEQ ID NO: 42)
5′ ACTACAGGTGTCCACTCCGAGGTGCAGCTGGTGGA 3′(sense);
bnIgG20L1,
(SEQ ID NO: 43)
5′ GTGTAAGCTTACCATGGGTGTGCCCACTCAGGTCCTGGGGT 3′
(sense);
bnIgG20H1,
(SEQ ID NO: 44)
5′ GTGTTCTAGAGCCGCCACCATGGAATGGAGCTGGGTCTTTC 3′
(sense).

For the generation of 2H3 mAb, the gene fragments of VL+CL, VH+CH1 and Fc domains were amplified from anti-ROR1 2H3 Fab with primer pairs ROR1-2H3-LC-FP/ROR1-2H3-LC-RP, ROR1-2H3-HC-FP/ROR1-2H3-CH1-RP, and ROR1-2H3-FC-FP/ROR1-2H3-FC-RP, respectively. The PCR products were fused to the 3′ end of H leader and L leader by overlapping PCR using the primer pairs bnIgG20H1/ROR1-2H3-CH1-RP and bnIgG20L1/ROR1-2H3-LC-RP, respectively. For the full-length heavy chain, the PCR products were fused with Fc domain by overlapping PCR using the primer pairs bnIgG20H1/ROR1-2H3-FC-RP. The heavy chain gene fragment was digested with XbaI and BamHI and cloned into the pDin1 vector. The light chain gene fragment was then further cloned into the construct containing the heavy chain insert via the HindIII and SacI restriction sites.

The 3A3 mAb was generated by using a similar protocol. The gene fragments of VL+CL, VH+CH1 and Fc domains were amplified from anti-ROR1 3A3 Fab with primer pairs ROR1-3A3-LC-FP/ROR1-2H3-LC-RP, ROR1-3A3-HC-FP/ROR1-2H3-CH1-RP, and ROR1-2H3-FC-FP/ROR1-2H3-FC-RP, respectively. The PCR products were fused to the 3′ end of H leader and L leader by overlapping PCR using the primer pairs bnIgG20H1/ROR1-2H3-CH1-RP and bnIgG20L1/ROR1-3A3-LC-RP, respectively. For the full-length heavy chain, the PCR products were fused with Fc domain by overlapping PCR using the primer pairs bnIgG20H1/ROR1-3A3-FC-RP. The heavy chain gene fragment was digested with XbaI and BamHI and cloned into the pDin1 vector. The light chain gene fragment was then further cloned into the construct containing the heavy chain insert via the HindIII and SacI restriction sites.

### Protein Expression, Purification and Initial Characterization

2H3 and 3A3 monoclonal antibodies were expressed in either 293FS or CHO-S cells. The plasmids and transfection agent PEI were mixed at ratio 1:3 and then added into 293FS or CHO-S cell culture. The cells were continued to grow for 5-7 days after transfection. The cell culture was harvested by centrifugation at 8000 rpm for 20 min. The culture supernatant containing target proteins were loaded onto Protein A Sepharose 4 Fast Flow column chromatography (GE Healthcare), and purified according to the manufacturer's instructions.

The purified proteins were subjected to SDS-PAGE. On a non-reducing SDS-PAGE, both mAbs display an apparent molecular weight (aMW) of approximately 150 kDa. On a reducing SDS-PAGE, the heavy chain and light chain have apparent molecular weight of approximately 55 kDa and 30 kDa, respectively (data not shown). The amino acid sequences of light chain variable region (VL) and heavy chain variable region (VH) of 2H3 and 3A3 monoclonal antibodies are shown in Table 1. The CDR sequences of the antibodies according to the Kabat system are shown in Table 2. The whole heavy chain and light chain sequences of the antibodies are shown in Table 3.

TABLE 1
VL and VH sequences of 2H3 and 3A3 monoclonal antibodies

| | | |
|---|---|---|
| 2H3 mAb VL | EIVLTQSPATLSLSPGERATLSCRASQSVSSYLAWY | SEQ ID NO: 1 |
| | QQKPGQAPRLLIYDASNRATGIPARFSGSGSGTDFT | |
| | LTISSLEPEDFAVYYCQQRSNWPLTFGGGTKVEIK | |
| 2H3 mAb VH | QVQLVQSGAEVKKTGSSVKVSCKASGYTFTYRYLHW | SEQ ID NO: 6 |
| | VRQAPGQALEWMGWITPFNGNTNYAQKFQDRVTITR | |
| | DRSMSTAYMELSSLRSEDTAMYYCARSGPRGDYVLD | |
| | YWGQGTLVTVSS | |
| 3A3 mAb VL | DVVMTQSPLSLPVTPGEPASISCRSSQSLLQSNGYN | SEQ ID NO: 11 |
| | YVEWFLQKPGQSPQLLIYLGSYRASGVPDRFSGSGS | |
| | GTDFTLKISRVEAEDVGVYYCMQGTHWPLFTFGPGT | |
| | KVDIK | |
| 3A3 mAb VH | EVQLVESGGGVVQPGRSLRLSCAASGFTFSSYGMHW | SEQ ID NO: 16 |
| | VRQAPGKGLEWVAVISYDGSNKYYADSVKGRFTISR | |
| | DNSKNTLYLQMNSLRAEDTAVYYCAKDLDYSLWFDP | |
| | WGQGTLVTVSS | |

TABLE 2
CDR sequences of 2H3 and 3A3
monoclonal antibodies

| | 2H3 mAb | 3A3 mAb |
|---|---|---|
| LCDR1 | RASQSVSSYLA (SEQ ID | RSSQSLLQSNGYNYVE |
| | NO: 2) | (SEQ ID NO: 12) |
| LCDR2 | DASNRAT (SEQ ID NO: | LGSYRAS (SEQ ID |
| | 3) | NO: 13) |
| LCDR3 | QQRSNWPLT (SEQ ID | MQGTHWPLFT (SEQ |
| | NO: 4) | ID NO: 14) |
| HCDR1 | GYTFTYR (SEQ ID NO: | GFTFSSY (SEQ ID |
| | 7) | NO: 17) |
| HCDR2 | TPFNGN (SEQ ID NO: | SYDGSN (SEQ ID |
| | 8) | NO: 18) |
| HCDR3 | SGPRGDYVLDY (SEQ | DLDYSLWFDP (SEQ |
| | ID NO: 9) | ID NO: 19) |

TABLE 3
Heavy chain and light chain sequences of
2H3 and 3A3 monoclonal antibodies

| | | |
|---|---|---|
| 2H3 mAb | EIVLTQSPATLSLSPGERATLSCRASQSVSS | SEQ ID |
| light | YLAWYQQKPGQAPRLLIYDASNRATGIPARF | NO: 5 |
| chain | SGSGSGTDFTLTISSLEPEDFAVYYCQQRSN | |
| | WPLTFGGGTKVEIKRTVAAPSVFIFPPSDEQ | |
| | LKSGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| 2H3 mAb | QVQLVQSGAEVKKTGSSVKVSCKASGYTFTY | SEQ ID |
| heavy | RYLHWVRQAPGQALEWMGWITPFNGNTNYAQ | NO: 10 |
| chain | KFQDRVTITRDRSMSTAYMELSSLRSEDTAM | |
| | YYCARSGPRGDYVLDYWGQGTLVTVSSASTK | |
| | GPSVFPLAPSSKSTSGGTAALGCLVKDYFPE | |
| | PVTVSWNSGALTSGVHTFPAVLQSSGLYSLS | |
| | SVVTVPSSSLGTQTYICNVNHKPSNTKVDKK | |
| | VEPKSCDKTHTCPPCPAPELLGGPSVFLFPP | |
| | KPKDTLMISRTPEVTCVVVDVSHEDPEVKFN | |
| | WYVDGVEVHNAKTKPREEQYNSTYRVVSVLT | |
| | VLHQDWLNGKEYKCKVSNKALPAPIEKTISK | |
| | AKGQPREPQVYTLPPSRDELTKNQVSLTCLV | |
| | KGFYPSDIAVEWESNGQPENNYKTTPPVLDS | |
| | DGSFFLYSKLTVDKSRWQQGNVFSCSVMHEA | |
| | LHNHYTQKSLSLSPGK | |
| 3A3 mAb | DVVMTQSPLSLPVTPGEPASISCRSSQSLLQ | SEQ ID |
| light | SNGYNYVEWFLQKPGQSPQLLIYLGSYRASG | NO: 15 |
| chain | VPDRFSGSGSGTDFTLKISRVEAEDVGVYYC | |
| | MQGTHWPLFTFGPGTKVDIKRTVAAPSVFIF | |
| | PPSDEQLKSGTASVVCLLNNFYPREAKVQWK | |
| | VDNALQSGNSQESVTEQDSKDSTYSLSSTLT | |
| | LSKADYEKHKVYACEVTHQGLSSPVTKSFNR | |
| | GEC | |
| 3A3 mAb | EVQLVESGGGVVQPGRSLRLSCAASGFTFSS | SEQ ID |
| heavy | YGMHWVRQAPGKGLEWVAVISYDGSNKYYAD | NO: 20 |
| chain | SVKGRFTISRDNSKNTLYLQMNSLRAEDTAV | |
| | YYCAKDLDYSLWFDPWGQGTLVTVSSASTKG | |
| | PSVFPLAPSSKSTSGGTAALGCLVKDYFPEP | |
| | VTVSWNSGALTSGVHTFPAVLQSSGLYSLSS | |
| | VVTVPSSSLGTQTYICNVNHKPSNTKVDKKV | |
| | EPKSCDKTHTCPPCPAPELLGGPSVFLFPPK | |
| | PKDTLMISRTPEVTCVVVDVSHEDPEVKFNW | |
| | YVDGVEVHNAKTKPREEQYNSTYRVVSVLTV | |
| | LHQDWLNGKEYKCKVSNKALPAPIEKTISKA | |
| | KGQPREPQVYTLPPSRDELTKNQVSLTCLVK | |
| | GFYPSDIAVEWESNGQPENNYKTTPPVLDSD | |
| | GSFFLYSKLTVDKSRWQQGNVFSCSVMHEAL | |
| | HNHYTQKSLSLSPGK | |

### Example 3. Construction and Initial Characterization of Anti-ROR1 Bispecific Antibodies

Bispecific T cell engager (BiTE) is a format of bispecific antibodies which guide cytotoxic T cells to kill cancer cells by simultaneously binding to a tumor antigen and a T cell antigen, such as CD3 molecule on T cell surface. HBiTE as described in PCT application No. PCT/US2018/016524 (which is incorporated herein by reference in its entirety) is a specific form of BiTE, in which the light chain, from N-terminus to C-terminus, comprises an anti-target VL domain, an anti-CD3 VL-CL and a monomeric human IgG1 Fc (e.g., mFc7.2). The heavy chain, from N-terminus to C-terminus, comprises an anti-target VH domain, an anti-CD3 VH-CH1 and a monomeric human IgG1 Fc (e.g., mFc7.2). Monomeric Fc7.2 contains two amino acid mutations (T366L and Y407H) capable of decreasing Fc homodimerization. To generate ROR1×CD3 HBiTE, VL and VH domains of the above anti-ROR1 antibodies were fused to the N-terminus of VL and VH domains of anti-CD3 Fab via a (G4S)3 linker, respectively. The anti-CD3 Fab is further fused to the N terminus of mFc7.2. The light chain and heavy chain were constructed into the vector pDin1 for mammalian cell expression. Construction and initial characterization of the two bispecific antibodies targeting ROR1 and CD3 were performed as follow.

### Cloning of Bispecific Antibodies Targeting ROR1 and CD3

To generate constructs of ROR1 bispecific antibodies, following primers were used:

bnIgG20L1,
(SEQ ID NO: 43)
5′ GTGTAAGCTTACCATGGGTGTGCCCACTCAGGTCCTGGGGT 3′
(sense);
ROR1-3A3 VL-forward,
(SEQ ID NO: 45)
5′ ACTACAGGTGTCCACTCCGATGTTGTGATGACTC 3′(sense);
ROR1-3A3 VL-reverse,
(SEQ ID NO: 46)
5′ GGGGGATCCTTTGATATCCACTTTGGTC 3′(antisense);
bnIgG20H1,
(SEQ ID NO: 44)
5′ GTGTTCTAGAGCCGCCACCATGGAATGGAGCTGGGTCTTTC 3′
(sense);
ROR1-3A3 VH-forward,
(SEQ ID NO: 47
5′ GGCTTACAGATGCCAGATGTGAGGTGCAGCTGGTG 3′(sense);
ROR1-3A3 VH-reverse,
(SEQ ID NO: 48)
5′ GATAGAGCTCCCTCCACCTGAGGAGACGGTGAC 3′
(antisense);
ROR1-2H3 VL-forward,
(SEQ ID NO: 49)
5′ ACTACAGGTGTCCACTCCGAAATTGTGTTGAC 3′(sense);
ROR1-2H3 VL-reverse,
(SEQ ID NO: 50)
5′ GGGGGATCCTTTGATCTCCACCTTG 3′(antisense);
ROR1-2H3 VH-forward,
(SEQ ID NO: 51)
5′ GGCTTACAGATGCCAGATGTCAGGTGCAGCTGGTAC 3′(sense);
ROR1-2H3 VH-reverse,
(SEQ ID NO: 52)
5′ GATAGAGCTCCCTCCACCTGAAGAGACGGTGACCAG 3′
(antisense).

For the generation of 2H3 HBiTE, the gene fragments of VL and VH domains were amplified from anti-ROR1 2H3 Fab with primer pairs ROR1-2H3 VL-forward/ROR1-2H3 VL-reverse and ROR1-2H3 VH-forward/ROR1-2H3 VH-reverse, respectively. The PCR products were fused to the 3′ end of H leader and L leader by overlapping PCR using the primer pairs bnIgG20H1/ROR1-2H3 VL-reverse and bnIgG20L1/ROR1-2H3 VH-reverse, respectively. The H leader-VL gene fragment was digested with XbaI and BamHI and cloned into a modified pDin1 vector for the expression of HBiTE, which contains an anti-CD3 hSP34 Fab and a complete Fc fragment. The L leader-VH gene fragment was then further cloned into the construct containing the H leader-VL insert via the HindIII and SacI restriction sites.

The 3A3 HBiTE was generated by using a similar protocol to 2H3 HBiTE. The gene fragments of VL and VH domains were amplified from anti-ROR1 3A3 Fab with primer pairs ROR1-3A3 VL-forward/ROR1-3A3 VL-reverse and ROR1-3A3 VH-forward/ROR1-3A3 VH-reverse, respectively. The PCR products were fused to the 3′ end of H leader and L leader by overlapping PCR using the primer pairs bnIgG20H1/ROR1-3A3 VL-reverse and bnIgG20L1/ROR1-3A3 VH-reverse, respectively. The H leader-VL gene fragment was digested with XbaI and BamHI and cloned into the HBiTE derived pDin1 vector containing an anti-CD3 hSP34 Fab and a complete Fc fragment. The L leader-VH gene fragment was then further cloned into the construct containing the H leader-VL insert via the HindIII and SacI restriction sites.

### Protein Expression, Purification and Initial Characterization

Bispecific antibodies were expressed in either 293FS or CHO-S cells. The plasmids and transfection agent PEI were mixed at ratio 1:3 and then added into 293FS or CHO-S cell culture. The cells were continued to grow for 5-7 days after transfection. The cell culture was harvested by centrifugation at 8000 rpm for 20 min. The culture supernatant containing target proteins were loaded onto Protein A Sepharose 4 Fast Flow column chromatography (GE Healthcare), and purified according to the manufacturer's instructions.

The purified proteins were subjected to SDS-PAGE. On a non-reducing SDS-PAGE, both HBiTEs display an apparent molecular weight (aMW) of approximately 120 kDa. On a reducing SDS-PAGE, the heavy chain and light chain are close to each other with an apparent molecular weight of approximately 62 kDa (data not shown). The amino acid sequences of light chain variable region (VL) and heavy chain variable region (VH) of 2H3 and 3A3 bispecific antibodies (HBiTEs) are shown in Table 4. The CDR sequences of the antibodies according to the Kabat system are shown in Table 5. The heavy chain and light chain sequences of the antibodies are shown in Table 6.

TABLE 4
VL and VH sequences of 2H3 and
3A3 bispecific antibodies

| | | | |
|---|---|---|---|
| 2H3 | VL | EIVLTQSPATLSLSPGERATLSCRASQ | SEQ ID |
| HBiTE | against | SVSSYLAWYQQKPGQAPRLLIYDASNR | NO: 1 |
| | ROR1 | ATGIPARFSGSGSGTDFTLTISSLEPE | |
| | | DFAVYYCQQRSNWPLTFGGGTKVEIK | |
| | VL | EIVVTQSPATLSVSPGERATLSCRSST | SEQ ID |
| | against | GAVTTSNYANWVQQKPGQAPRGLIGGA | NO: 21 |
| | CD3 | NKRAPGVPARFSGSLSGDEATLTISSL | |
| | | QSEDFAVYYCALWYSNLWVFGQGTKLE | |
| | | IK | |
| | VH | QVQLVQSGAEVKKTGSSVKVSCKASGY | SEQ ID |
| | against | TFTYRYLHWVRQAPGQALEWMGWITPF | NO: 6 |
| | ROR1 | NGNTNYAQKFQDRVTITRDRSMSTAYM | |
| | | ELSSLRSEDTAMYYCARSGPRGDYVLD | |
| | | YWGQGTLVTVSS | |
| | VH | EVQLVESGGGLVQPGGSLRLSCAASGF | SEQ ID |
| | against | TFNTYAMNWVRQAPGKGLEWVARIRSK | NO: 26 |
| | CD3 | YNNYATYYADSVKGRFTISRDDSKNTL | |
| | | YLQMNSLRAEDTAVYYCARHGNFGSSY | |
| | | VSYFAYWGQGTTVTVSS | |
| 3A3 | VL | DVVMTQSPLSLPVTPGEPASISCRSSQ | SEQ ID |
| | against | SLLQSNGYNYVEWFLQKPGQSPQLLIY | NO: 11 |
| | ROR1 | LGSYRASGVPDRFSGSGSGTDFTLKIS | |
| | | RVEAEDVGVYYCMQGTHWPLFTFGPGT | |
| | | KVDIK | |
| | VL | EIVVTQSPATLSVSPGERATLSCRSST | SEQ ID |
| | against | GAVTTSNYANWVQQKPGQAPRGLIGGA | NO: 21 |
| | CD3 | NKRAPGVPARFSGSLSGDEATLTISSL | |
| | | QSEDFAVYYCALWYSNLWVFGQGTKLE | |
| | | IK | |
| | VH | EVQLVESGGGVVQPGRSLRLSCAASGF | SEQ ID |
| | against | TFSSYGMHWVRQAPGKGLEWVAVISYD | NO: 16 |
| | ROR1 | GSNKYYADSVKGRFTISRDNSKNTLYL | |
| | | QMNSLRAEDTAVYYCAKDLDYSLWFDP | |
| | | WGQGTLVTVSS | |
| | VH | EVQLVESGGGLVQPGGSLRLSCAASGF | SEQ ID |
| | against | TFNTYAMNWVRQAPGKGLEWVARIRSK | NO: 26 |
| | CD3 | YNNYATYYADSVKGRFTISRDDSKNTL | |
| | | YLQMNSLRAEDTAVYYCARHGNFGSSY | |
| | | VSYFAYWGQGTTVTVSS | |

TABLE 5
CDR sequences of 2H3 and
3A3 bispecific antibodies

| | 2H3 HBITE | 3A3 HBITE |
|---|---|---|
| LCDR1 against | RASQSVSSYLA | RSSQSLLQSNGYNYVE |
| ROR1 | (SEQ ID NO: 2) | (SEQ ID NO: 12) |
| LCDR2 against | DASNRAT (SEQ | LGSYRAS (SEQ ID |
| ROR1 | ID NO: 3) | NO: 13) |
| LCDR3 against | QQRSNWPLT (SEQ | MQGTHWPLFT (SEQ |
| ROR1 | ID NO: 4) | ID NO: 14) |
| HCDR1 against | GYTFTYR (SEQ | GFTFSSY (SEQ ID |
| ROR1 | ID NO: 7) | NO: 17) |
| HCDR2 against | TPFNGN (SEQ ID | SYDGSN (SEQ ID |
| ROR1 | NO: 8) | NO: 18) |
| HCDR3 against | SGPRGDYVLDY | DLDYSLWFDP (SEQ |
| | (SEQ ID NO: 9) | ID NO: 19) |
| LCDR1 against | RSSTGAVTTSNYAN | RSSTGAVTTSNYAN |
| CD3 | (SEQ ID NO: 22) | (SEQ ID NO: 22) |
| LCDR2 against | GANKRAP (SEQ | GANKRAP (SEQ ID |
| CD3 | ID NO: 23) | NO: 23) |
| LCDR3 against | ALWYSNLWV (SEQ | ALWYSNLWV (SEQ |
| CD3 | ID NO: 24) | ID NO: 24) |
| HCDR1 against | GFTFNTY (SEQ | GFTFNTY (SEQ ID |
| CD3 | ID NO: 27) | NO: 27) |
| HCDR2 against | RSKYNNYA (SEQ | RSKYNNYA (SEQ |
| CD3 | ID NO: 28) | ID NO: 28) |
| HCDR3 against | HGNFGSSYVSYFAY | HGNFGSSYVSYFAY |
| CD3 | (SEQ ID NO: 29) | (SEQ ID NO: 29) |

TABLE 6
Heavy chain and light chain sequences of
2H3 and 3A3 bispecific antibodies

| | | |
|---|---|---|
| 2H3 | EIVLTQSPATLSLSPGERATLSCRASQSVSSYLAWY | SEQ ID |
| HBITE | QQKPGQAPRLLIYDASNRATGIPARFSGSGSGTDFT | NO: 25 |
| light | LTISSLEPEDFAVYYCQQRSNWPLTFGGGTKVEIKG | |
| chain | GGGSGGGGSGGGGSEIVVTQSPATLSVSPGERATLS | |
| | CRSSTGAVTTSNYANWVQQKPGQAPRGLIGGANKRA | |
| | PGVPARFSGSLSGDEATLTISSLQSEDFAVYYCALW | |
| | YSNLWVFGQGTKLEIKRTVAAPSVFIFPPSDEQLKS | |
| | GTASVVCLLNNFYPREAKVQWKVDNALQSGNSQESV | |
| | TEQDSKDSTYSLSSTLTLSKADYEKHKVYACEVTHQ | |
| | GLSSPVTKSFNRGECPPCPAPELLGGPSVFLFPPKP | |
| | KDTLMISRTPEVTCVVVDVSHEDPEVKFNWYVDGVE | |
| | VHNAKTKPREEQYNSTYRVVSVLTVLHQDWLNGKEY | |
| | KCKVSNKALPAPIEKTISKAKGQPREPQVYTLPPSR | |
| | DELTKNQVSLLCLVKGFYPSDIAVEWESNGQPENNY | |
| | KTTPPVLDSDGSFFLHSKLTVDKSRWQQGNVFSCSV | |
| | MHEALHNHYTQKSLSLSPGK | |
| 2H3 | QVQLVQSGAEVKKTGSSVKVSCKASGYTFTYRYLHW | SEQ ID |
| HBITE | VRQAPGQALEWMGWITPFNGNTNYAQKFQDRVTITR | NO: 30 |
| heavy | DRSMSTAYMELSSLRSEDTAMYYCARSGPRGDYVLD | |
| chain | YWGQGTLVTVSSGGGSSGGGGSGGGGSEVQLVESGG | |
| | GLVQPGGSLRLSCAASGFTFNTYAMNWVRQAPGKGL | |
| | EWVARIRSKYNNYATYYADSVKGRFTISRDDSKNTL | |
| | YLQMNSLRAEDTAVYYCARHGNFGSSYVSYFAYWGQ | |
| | GTTVTVSSASTKGPSVFPLAPSSKSTSGGTAALGCL | |
| | VKDYFPEPVTVSWNSGALTSGVHTFPAVLQSSGLYS | |
| | LSSVVTVPSSSLGTQTYICNVNHKPSNTKVDKKVEP | |
| | KSCPPCPAPELLGGPSVFLFPPKPKDTLMISRTPEV | |
| | TCVVVDVSHEDPEVKFNWYVDGVEVHNAKTKPREEQ | |
| | YNSTYRVVSVLTVLHQDWLNGKEYKCKVSNKALPAP | |
| | IEKTISKAKGQPREPQVYTLPPSRDELTKNQVSLLC | |
| | LVKGFYPSDIAVEWESNGQPENNYKTTPPVLDSDGS | |
| | FFLHSKLTVDKSRWQQGNVFSCSVMHEALHNHYTQK | |
| | SLSLSPGK | |
| 3A3 | DVVMTQSPLSLPVTPGEPASISCRSSQSLLQSNGYN | SEQ ID |
| HBITE | YVEWFLQKPGQSPQLLIYLGSYRASGVPDRESGSGS | NO: 31 |
| light | GTDFTLKISRVEAEDVGVYYCMQGTHWPLFTFGPGT | |
| chain | KVDIKGGGGSGGGGSGGGGSEIVVTQSPATLSVSPG | |
| | ERATLSCRSSTGAVTTSNYANWVQQKPGQAPRGLIG | |
| | GANKRAPGVPARFSGSLSGDEATLTISSLQSEDFAV | |
| | YYCALWYSNLWVFGQGTKLEIKRTVAAPSVFIFPPS | |
| | DEQLKSGTASVVCLLNNFYPREAKVQWKVDNALQSG | |
| | NSQESVTEQDSKDSTYSLSSTLTLSKADYEKHKVYA | |
| | CEVTHQGLSSPVTKSFNRGECPPCPAPELLGGPSVF | |
| | LFPPKPKDTLMISRTPEVTCVVVDVSHEDPEVKFNW | |
| | YVDGVEVHNAKTKPREEQYNSTYRVVSVLTVLHQDW | |
| | LNGKEYKCKVSNKALPAPIEKTISKAKGQPREPQVY | |
| | TLPPSRDELTKNQVSLLCLVKGFYPSDIAVEWESNG | |
| | QPENNYKTTPPVLDSDGSFFLHSKLTVDKSRWQQGN | |
| | VFSCSVMHEALHNHYTQKSLSLSPGK | |
| 3A3 | EVQLVESGGGVVQPGRSLRLSCAASGFTFSSYGMHW | SEQ ID |
| HBITE | VRQAPGKGLEWVAVISYDGSNKYYADSVKGRFTISR | NO: 32 |
| heavy | DNSKNTLYLQMNSLRAEDTAVYYCAKDLDYSLWFDP | |
| chain | WGQGTLVTVSSGGGSSGGGGSGGGGSEVQLVESGGG | |
| | LVQPGGSLRLSCAASGFTFNTYAMNWVRQAPGKGLE | |
| | WVARIRSKYNNYATYYADSVKGRFTISRDDSKNTLY | |
| | LQMNSLRAEDTAVYYCARHGNFGSSYVSYFAYWGQG | |
| | TTVTVSSASTKGPSVFPLAPSSKSTSGGTAALGCLV | |
| | KDYFPEPVTVSWNSGALTSGVHTFPAVLQSSGLYSL | |
| | SSVVTVPSSSLGTQTYICNVNHKPSNTKVDKKVEPK | |
| | SCPPCPAPELLGGPSVFLFPPKPKDTLMISRTPEVT | |
| | CVVVDVSHEDPEVKFNWYVDGVEVHNAKTKPREEQY | |
| | NSTYRVVSVLTVLHQDWLNGKEYKCKVSNKALPAPI | |
| | EKTISKAKGQPREPQVYTLPPSRDELTKNQVSLLCL | |
| | VKGFYPSDIAVEWESNGQPENNYKTTPPVLDSDGSF | |
| | FLHSKLTVDKSRWQQGNVFSCSVMHEALHNHYTQKS | |
| | LSLSPGK | |

### Example 4. Binding Affinity of Anti-ROR1 Monoclonal Antibodies to ROR1

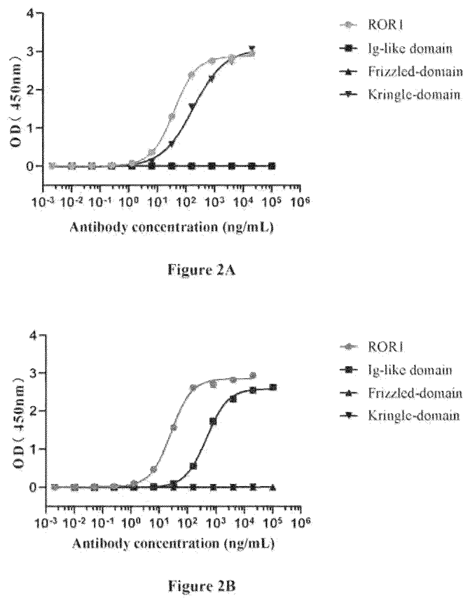

ELISA was performed according to standard protocols, to determine binding affinity of anti-ROR1 mAB 2H3 and 3A3 to full extracellular domain and individual extracellular Ig-like domain, frizzled domain, and kringle domain of human ROR1. Briefly, recombinant human ROR1 (AcroBiosystems) were coated on Corning EIA/RIA high-binding 96-well plates (Corning Inc.) at 50 ng per well overnight at 4° C. and blocked with 3% nonfat milk in PBS (pH7.4). Fivefold serially diluted biotinylated antibodies were added and incubated at room temperature for 2 h. The plates were washed with PBS containing 0.05% Tween 20. Bound antibodies were detected by HRP-conjugated streptavidin (Sino Biological). The assay was developed at room temperature with TMB substrate (Solarbio) and monitored at 450 nm with a microplate reader. The half-maximal binding (EC₅₀) was calculated by fitting the data to the Langmuir adsorption isotherm. The results were shown in FIG. 2.

The results indicate that the 2H3 mAb can bind to full extracellular domain and kringle domain with EC50 of 0.2 nM and 1.2 nM, respectively, suggesting binding epitope of the 2H3 mAb is located in kringle domain of ROR1 (FIG. 2A); while the 3A3 mAb can bind to full extracellular domain and Ig-like domain with EC50 of 0.18 nM and 3.1 nM, respectively, suggesting the 3A3 mAb has high binding affinity to Ig-like domain of ROR1 (FIG. 2B).

### Example 5. Binding Test of Anti-ROR1 Monoclonal Antibodies to Cell Surface-Associated ROR1 in Various Cancer Cell Lines

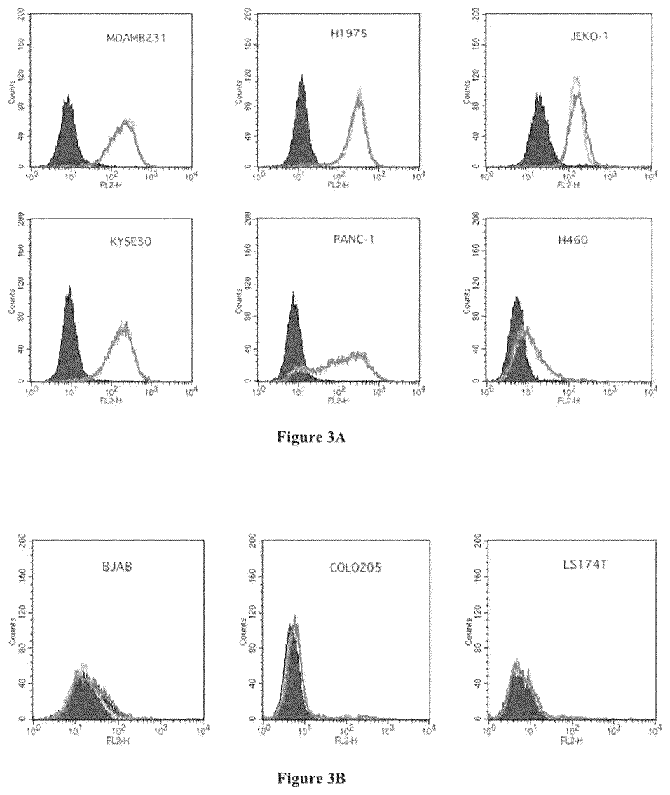

To measure binding ability of the anti-ROR1 mAbs 2H3 and 3A3 to cell surface-associated ROR1, flow cytometry was carried out for multiple cancer cell lines including MDA-MB-231, H1975, JEKO-1, KYSE-30, PANC-1, H460, BJAB, COLO205, and LS174T. About 5×10⁵ cells were incubated with antibodies (10 μg/ml) on ice for 1 h. The cells were washed once with PBS containing 0.1% bovine serum albumin (PBSA) and resuspended in 100 μl PBSA. Then 1 μl anti-human IgG (Fc-specific)-FITC conjugate (Sigma) was added and incubated for 30 min. The cells were washed once with PBSA and then used for flow cytometry analysis. The results were shown in FIG. 3.

The results indicate that mAbs 2H3 and 3A3 have highly similar binding ability to these cell lines. The both mAbs can bind well to MDA-MB-231, H1975, JEKO-1, KYSE-30, PANC-1, H460 cell lines (FIG. 3A), while showing no binding to cell lines BJAB, COLO205, and LS174T (FIG. 3B). This suggests that 2H3 and 3A3 mAbs have superior binding ability to ROR1 positive cancer cell lines and can be used to kill multiple cancer cells expressing ROR1.

### Example 6. Binding Affinity of Bispecific Antibodies Targeting ROR1 and CD3 to ROR1 and CD3

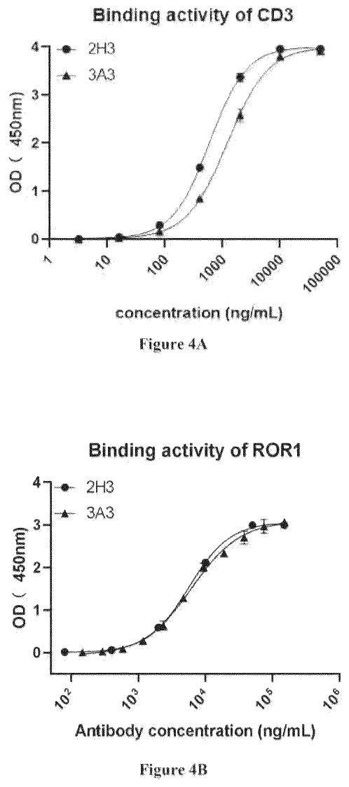

To determine binding affinity of 2H3 and 3A3 bispecific antibodies to both human ROR1 and CD3, ELISA was performed as described in Example 5. The results were shown in FIG. 4. The results indicate that the 2H3 and 3A3 bispecific antibodies bind to human recombinant CD3 with EC50 of 4.9 nM and 10 nM, respectively (FIG. 4A), and bind to ROR1 with EC50 of 47 nM and 51 nM, respectively (FIG. 4B).

### Example 7. Binding Affinity of Bispecific Antibodies Targeting ROR1 and CD3 to Cancer Cell Lines

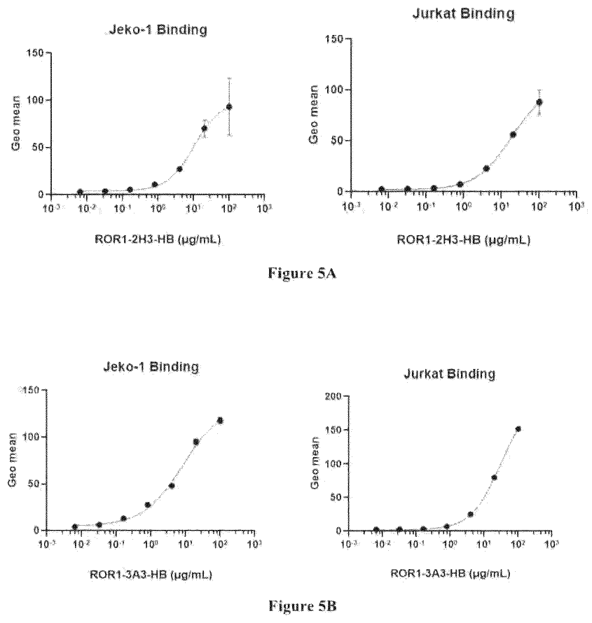

To determine binding affinity of bispecific antibodies 2H3 and 3A3 to cancer cell lines, flow cytometry was carried out for ROR1 expressing cell line JEKO-1 and CD3 positive Jurkat cell line as described in Example 6. The results were shown in FIG. 5.

The results indicate that the 2H3 bispecific antibodies binds to ROR1 expressing MCL cell line with EC50 of 86.5 nM and binds to CD3 expressing Jurkat cell line with EC50 of 154.9 nM (FIG. 5A), while The 3A3 bispecific antibodies binds to ROR1 expressing MCL cell line with EC50 of 71.4 nM and binds to CD3 expressing Jurkat cell line with EC50 of 251.8 nM (FIG. 5B).

### Example 8. Bispecific Antibodies Targeting ROR1 and CD3-Mediated Killing of Human Cancer Cell Lines

Bispecific T cell engager can simultaneously bind to specific tumor antigen and T cell antigen (e.g., CD3 molecular on T cell surface) causing aggregation and activation of T cells, eventually leading to the killing of tumor cells. To evaluate killing efficiency of the bispecific antibodies targeting ROR1 and CD3 in form of HBiTE, four ROR1 expressing cell lines including JEKO-1, MDA-MB-231, SK-HEP-1, and PANC-1 were used as target cells.

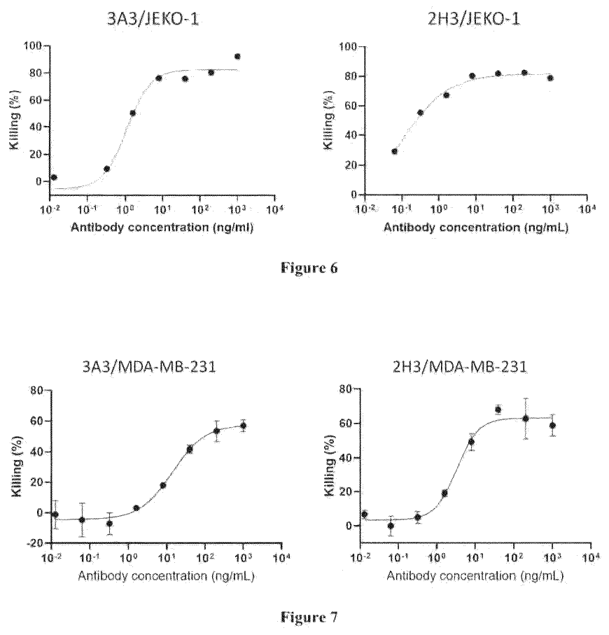

For suspension cell line (JEKO-1), flow cytometry was performed to detect CFSE Labeled JEKO-1 activity. A single-cell suspension of JEKO-1 was collected in 50 ml centrifuge tube. Cells were washed two times with PBS to remove any serum and resuspended with PBS at a density of 5×10⁶/ml. CFSE was added into cell suspension at a final concentration of 0.5 μM. 10 minutes after incubation at room temperature in the dark, 4-5 fold volumes of cold complete media (containing ≥10% serum) was added to stop labeling. Cells were washed 3 times using complete media. 2×10⁴ target cells were seeded in 100 μl RPMI 1640 complete medium for each well. At the same day, 10⁵ PBMC in 50 μl RPMI 1640 complete medium were added into each well (a ratio of target cells to effector cells=1:5). Then, 50 μl antibodies 5-fold serially diluted from 1 μg/ml were added into each well. 48 h after incubation, the cells were processed following standard protocol for flow cytometry measurement. The results were shown in FIG. 6.

For adherent cell lines (DA-MB-231, SK-HEP-1, and PANC-1), 10⁴ target cells were seeded in 100 μl RPMI 1640 complete medium overnight. Meanwhile, frozen PBMC were revived and inoculated in 30 mL RPMI 1640 complete medium overnight. At the second day, 10⁵ PBMC in 50 μl RPMI 1640 complete medium were added (actual target cells:effector cells ratio=1:5 because target cells duplicate overnight). Then, 50 μl antibodies 5-fold serially diluted from 1 μg/ml were added into each well. 48 h after incubation, the medium was removed from target cells and 100 μl RPMI 1640 complete medium containing 10% CCK8 was added and incubated 30 minutes in CO₂ incubator. Cell killing activity was measured by using microplate reader according to the manufacturer's instructions. The results were shown in FIGS. 7-9.

As can be seen from FIG. 6, for human mantle cell lymphoma cell line JEKO-1, both 2H3 and 3A3 bispecific antibodies show potent killing ability against over 80% tumor cells in the presence of PBMC. EC50 values of 2H3 and 3A3 bispecific antibodies are 0.083 ng/ml and 1.098 ng/ml, respectively.

As can be seen from FIG. 7, for human breast cancer cell line MDA-MB-23, 2H3 and 3A3 bispecific antibodies also show potent killing efficiency against over 60% tumor cells in the presence of PBMC. EC50 values of 2H3 and 3A3 bispecific antibodies are 3.312 ng/ml and 13.99 ng/ml, respectively.

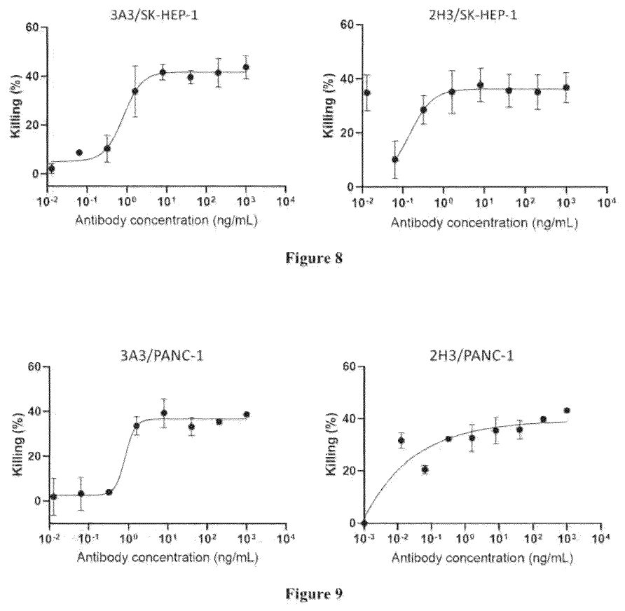

As can be seen from FIG. 8, for human hepatic adenocarcinoma cell line SK-HEP-1, both 2H3 and 3A3 bispecific antibodies show killing potency against around 40% tumor cells with EC50 values of 0.144 ng/ml and 0.805 ng/ml, respectively.

As can be seen from FIG. 9, for human pancreatic cancer cell line PANC-1, 2H3 and 3A3 bispecific antibodies show killing potency against around 40% tumor cells.

Taken together, both 2H3 and 3A3 bispecific antibodies have shown potent killing activity against multiple cancer cell lines, suggesting good potential for treating various cancers expressing ROR1.

### Example 9. Anti-ROR1 Monoclonal Antibodies Mediated ADCC Against Human Cancer Cell Line

Frozen NK cells were revived and cultured in RPMI1640 complete medium containing 20% FBS, 1% penicillin/streptomycin and 50 IU IL-2 overnight at 37° C. and 5% CO₂. Human colorectal cancer cell line HT29 cells were used as target cells and diluted to a concentration of 1.5×10⁵ cells/mL with the complete medium, and added to a 96-well plate at 100 μL/well and cultured overnight at 37° C. Anti-ROR1 monoclonal antibodies 2H3 Mab and 3A3 Mab were prepared to concentrations of 400 μg/mL, 40 μg/mL and 4 μg/mL, respectively, with RPMI1640 medium, and an IgG isotype antibody was used as negative control. The prepared antibody solutions were added to the 96-well plate containing target cells at 50 μL/well. NK cells were collected by centrifugation and diluted to 6×10⁵ cells/mL with the complete medium. 50 μL of NK cells were added to the 96-well plate. The final concentrations of the antibodies were 100 μg/mL, 10 μg/mL and 1 μg/mL, respectively. All culture plates were incubated at 37° C. for 72 h. Then, the original medium was removed and replaced with fresh medium containing 10% CCK-8 at 100 μL/well. The plates were incubated at 37° C. for about 30 min, and measured for OD values using a microplate reader at 450 nm (reference wavelength was 630 nm).

Killing efficiency was calculated by the following equation:

$$Cytotoxicity \% = ( {OD}_{Tumor + NK + 0 μ g / mL mab} - {OD}_{Tumor + NK + x μ g / mL mab} ) /$$

$${OD}_{Tumor + NK + 0 μ g / mL mab} \times 100 \% ,$$

in which x represents 1, 10 or 100.

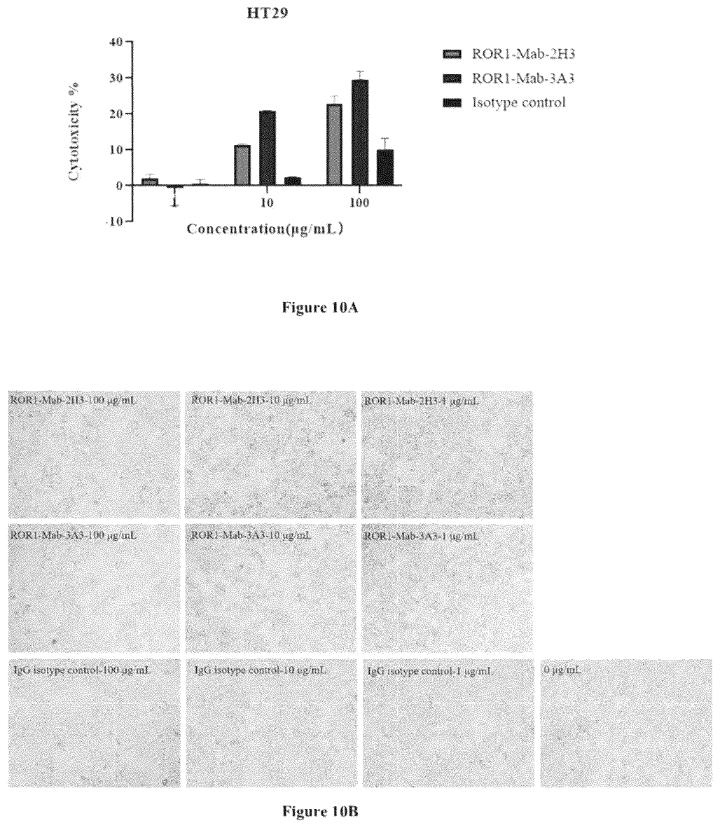

The ADCC killing of 2H3 Mab and 3A3 Mab against HT29 cells was shown in FIGS. 10A-10B. The results indicate that 2H3 Mab and 3A3 Mab mediate significantly increased ADCC killing against HT29 cells, compared with the control group, and the killing efficiency is dose-dependent. This suggests that the 2H3 Mab and 3A3 Mab have potent killing efficiency against cancer cell lines expressing ROR1.

### Example 10. Bispecific Antibodies Mediated Inhibition of Tumor Growth in Mice

To verify anti-tumor effect in vivo of 2H3 bispecific antibody, colon cancer cells LS174T-ROR1 over-expressing human ROR1 (1×10⁶ cells/mouse) and effector cells human PBMCs (1.5×10⁶ cells/mouse) were mixed and inoculated subcutaneously into the right axilla of B-NDG mice. At day 12 after the inoculation, tumor volume reached about 100 mm³, and the mice were grouped and dosed. In the experiment group, 10 μg/kg of 2H3 bispecific antibody (ROR1-2H3-HB) was injected intratumorally into mice for three times one week. Saline solution was used as a negative control. After one week of treatment, tumor volume was measured at day 12, 15, 17 and 19 after the inoculation. At day 19 after the inoculation, mice were sacrificed and tumor weight was measured.

To verify anti-tumor effect in vivo of 3A3 bispecific antibody, colon cancer cells LS174T-ROR1 over-expressing human ROR1 (2×10⁶ cells/mouse) and effector cells human PBMCs (2×10⁶ cells/mouse) were mixed and inoculated subcutaneously into the right axilla of B-NDG mice. At day 6 after the inoculation, tumor volume reached about 100 mm³, and the mice were grouped and dosed. In the experiment group, 10 μg/kg of 3A3 bispecific antibody (ROR1-3A3-HB) was injected intratumorally into mice for three times one week. Saline solution was used as a negative control. After one week of treatment, tumor volume was measured at day 6, 8, 12 and 15 after the inoculation. At day 15 after the inoculation, mice were sacrificed and tumor weight was measured.

Tumor growth inhibition (TGI) rate for tumor volume was calculated by using the following formula:

(Average tumor volume of control group−average tumor volume of experiment group)/average tumor volume of control group.

Tumor growth inhibition (TGI) rate for tumor weight was calculated by using the following formula:

(Average tumor weight of control group−average tumor weight of experiment group)/average tumor weight of control group.

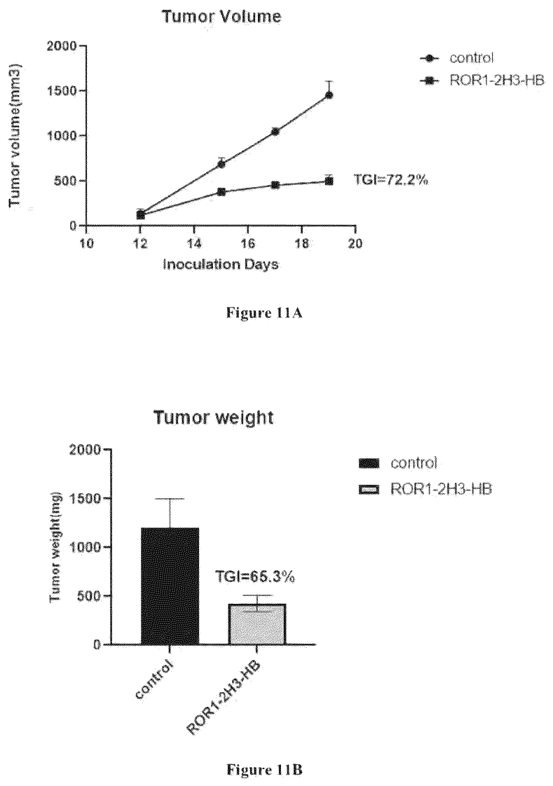

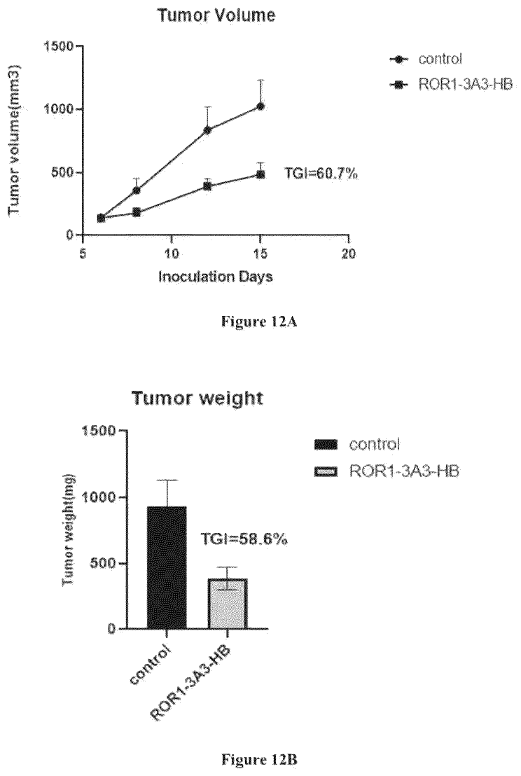

The results for 2H3 and 3A3 bispecific antibodies were shown in FIGS. 11A-11B and FIGS. 12A-12B, respectively.

The results show that 2H3 bispecific antibody significantly inhibits tumor growth in the mice model with tumor volume inhibition rate of 72.2% (FIG. 11A) and tumor weight inhibition rate of 65.3% (FIG. 11B); and 3A3 bispecific antibody significantly inhibits tumor growth in the mice model with tumor volume inhibition rate of 60.7% (FIG. 12A) and tumor weight inhibition rate of 58.6% (FIG. 12B).

In summary, the results have demonstrated that 2H3 and 3A3 bispecific antibodies can potently inhibit growth of the tumor cells expressing ROR1, suggesting its potential for treating ROR1 positive cancers.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. An antibody specifically binding to ROR1, or an antigen binding fragment thereof, comprising a light chain variable region (VL) and a heavy chain variable region (VH), wherein
(i) the VL comprises LCDRs 1-3 having the amino acid sequences as shown in SEQ ID NOs: 2-4 respectively, and the VH comprises HCDRs 1-3 having the amino acid sequences as shown in SEQ ID NOs: 7-9 respectively; or
(ii) the VL comprises LCDRs 1-3 having the amino acid sequences as shown in SEQ ID NOs: 12-14 respectively, and the VH comprises HCDRs 1-3 having the amino acid sequences as shown in SEQ ID NOs: 17-19 respectively.

2. The antibody or the antigen binding fragment thereof according to claim 1, wherein
(i) the VL comprises an amino acid sequence having at least 80%, at least 85%, at least 90%, at least 95%, at least 98%, at least 99%, or 100% sequence identity to SEQ ID NO: 1 and the VH comprises an amino acid sequence having at least 80%, at least 85%, at least 90%, at least 95%, at least 98%, at least 99%, or 100% sequence identity to SEQ ID NO: 6; or
(ii) the VL comprises an amino acid sequence having at least 80%, at least 85%, at least 90%, at least 95%, at least 98%, at least 99%, or 100% sequence identity to SEQ ID NO: 11 and the VH comprises an amino acid sequence having at least 80%, at least 85%, at least 90%, at least 95%, at least 98%, at least 99%, or 100% sequence identity to SEQ ID NO: 16.

3. The antibody or the antigen binding fragment thereof according to claim 1, wherein the antibody comprises
(i) a light chain comprising an amino acid sequence having at least 80%, at least 85%, at least 90%, at least 95%, at least 98%, at least 99%, or 100% sequence identity to SEQ ID NO: 5 and a heavy chain comprising an amino acid sequence having at least 80%, at least 85%, at least 90%, at least 95%, at least 98%, at least 99%, or 100% sequence identity to SEQ ID NO: 10; or
(ii) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20.

4. The antibody or the antigen binding fragment thereof according to claim 1, wherein the antibody is of an isotype selected from the group consisting of IgG, IgA, IgM, IgE and IgD.

5. The antibody or the antigen binding fragment thereof according to claim 1, wherein the antibody is of a subtype selected from the group consisting of IgG1, IgG2, IgG3, and IgG4.

6. The antibody or the antigen binding fragment thereof according to claim 1, wherein the antigen binding fragment is selected from the group consisting of Fab, Fab′, F(ab′)₂, Fd, Fd′, Fv, scFv, ds-scFv and dAb.

7. The antibody or the antigen binding fragment thereof according to claim 1, wherein the antibody is a monoclonal antibody, a bi-specific or a multi-specific antibody.

8. (canceled)

9. The antibody or the antigen binding fragment thereof according to claim 7, wherein the antibody is a bispecific antibody which further comprises a second antigen binding region binding to a second antigen.

10. The antibody or the antigen binding fragment thereof of according to claim 9, wherein the second antigen is a tumor associated antigen, an immune cell antigen, or a T-cell antigen.

11. (canceled)

12. The antibody or the antigen binding fragment thereof according to claim 10, wherein the T-cell antigen is selected from the group consisting of T cell receptor (TCR), CD3, CD4, CD8, CD16, CD25, CD28, CD44, CD62L, CD69, ICOS, 41-BB (CD137), and NKG2D or any combination thereof.

13. The antibody or the antigen binding fragment thereof according to claim 9, wherein the second antigen is CD3, and the second antigen binding region comprises a VL and a VH, wherein the VL comprises LCDRs 1-3 having the amino acid sequences as shown in SEQ ID NOs: 22-24 respectively, and the VH comprises HCDRs 1-3 having the amino acid sequences as shown in SEQ ID NOs: 27-29 respectively.

14. The antibody or the antigen binding fragment thereof according to claim 13, wherein the second antigen binding region comprises a VL comprising an amino acid sequence having at least 80%, at least 85%, at least 90%, at least 95%, at least 98%, at least 99%, or 100% sequence identity to SEQ ID NO: 21 and a VH comprising an amino acid sequence having at least 80%, at least 85%, at least 90%, at least 95%, at least 98%, at least 99%, or 100% sequence identity to SEQ ID NO: 26.

15. The antibody or the antigen binding fragment thereof according to claim 13, wherein the VL of the second antigen binding region is linked to the C-terminal of the VL of the antibody specifically binding to ROR-1, and the VH of the second antigen binding region is linked to the C-terminal of the VH of the antibody specifically binding to ROR-1.

16. The antibody or the antigen binding fragment thereof according to claim 15, wherein the VL of the second antigen binding region is linked to the VL of the antibody specifically binding to ROR-1 via a linker having the amino acid sequence as shown in SEQ ID NO: 33, and the VH of the second antigen binding region is linked to the VH of the antibody specifically binding to ROR-1 via a linker having the amino acid sequence as shown in SEQ ID NO: 34.

17. The antibody or the antigen binding fragment thereof according to claim 13, wherein the bispecific antibody comprises
(i) a light chain comprising an amino acid sequence having at least 80%, at least 85%, at least 90%, at least 95%, at least 98%, at least 99%, or 100% sequence identity to SEQ ID NO: 25 and a heavy chain comprising an amino acid sequence having at least 80%, at least 85%, at least 90%, at least 95%, at least 98%, at least 99%, or 100% sequence identity to SEQ ID NO: 30; or
(ii) a light chain comprising an amino acid sequence having at least 80%, at least 85%, at least 90%, at least 95%, at least 98%, at least 99%, or 100% sequence identity to SEQ ID NO: 31 and a heavy chain comprising an amino acid sequence having at least 80%, at least 85%, at least 90%, at least 95%, at least 98%, at least 99%, or 100% sequence identity to SEQ ID NO: 32.

18.-24. (canceled)

25. A nucleic acid comprising a nucleotide sequence encoding the antibody or the antigen binding fragment thereof according to claim 1.

26.-27. (canceled)

28. A pharmaceutical composition comprising (i) the antibody or the antigen binding fragment thereof according to claim 1; and (ii) a pharmaceutically acceptable carrier or adjuvant.

29. A antibody-drug conjugate, comprising the antibody or the antigen binding fragment thereof according to claim 1.

30. A method of treating cancer in a subject, comprising administering to the subject an effective amount of the antibody or the antigen binding fragment thereof according to claim 1.

31. The method according to claim 30, wherein the cancer is selected from the group consisting of breast cancer, lung cancer, ovarian cancer, colon cancer, liver cancer, esophageal cancer, pancreatic cancer, bladder cancer, prostate cancer, colorectal cancer, uterine cancer, cervical cancer, brain cancer, cervical cancer, gastric cancer, cholangiocarcinoma, chondrosarcoma, kidney cancer, thyroid cancer, skin cancer, lymphoma, myeloma, and leukemia, preferably selected from the group consisting of chronic lymphocytic leukemia (CLL), mantle cell lymphoma (MCL), B-cell acute lymphoblastic leukemia, T-cell acute lymphoblastic leukemia, Burkitt lymphoma, multiple myeloma, lung adenocarcinoma, non-small cell lung cancer (NSCLC), human esophageal squamous cell carcinoma, colonic adenocarcinoma, breast cancer, pancreatic cancer, bladder cancer, colorectal cancer, liver cancer, and ovarian cancer.

32.-34. (canceled)
